(19) United States
(12) Patent Application Publication
(10) Pub. No.: US 2022/0019284 A1
(43) Pub. Date: Jan. 20, 2022

(54) FEEDBACK FROM NEUROMUSCULAR ACTIVATION WITHIN VARIOUS TYPES OF VIRTUAL AND/OR AUGMENTED REALITY ENVIRONMENTS

(71) Applicant: Facebook Technologies, LLC, Menlo Park, CA (US)

(72) Inventors: Patrick KAIFOSH, New York, NY (US); Alexandre BARACHANT, Brooklyn, NY (US); Mason REMALEY, Santa Cruz, CA (US); Julien KILlAN, New York, NY (US); Kirak HONG, Fort Lee, NJ (US); Nathan DANIELSON, New York, NY (US); Qiushi MAO, Brooklyn, NY (US); Daniel WETMORE, New York, NY (US)

(21) Appl. No.: 17/293,472

(22) PCT Filed: Nov. 15, 2019
(86) PCT No.: PCT/US2019/061759

§ 371 Date: May 12, 2021

Related U.S. Application Data
(60) Provisional application No. 62/768,741, filed on Nov. 16, 2018.

(51) Int. Cl. G06F 3/01; G06F 3/16

(52) U.S. Cl. CPC G06F 3/015; G06F 3/16; G06F 3/016

(57) ABSTRACT

Computerized systems, methods, and computer-readable storage media storing code for the methods enable feedback to be provided to a user based on neuromuscular signals sensed from the user. One such system includes neuromuscular sensors and at least one computer processor. The sensors, which are arranged on one or more wearable devices, are configured to sense neuromuscular signals from the user. The at least one computer processor is or are programmed to process the neuromuscular signals using one or more inference models, and to provide feedback to the user based on one or both of: the processed neuromuscular signals and information derived from the processed neuromuscular signals. The feedback includes visual feedback of information relating to one or both of: a timing of an activation of at least one motor unit of the user and an intensity of the activation of the at least one motor unit of the user.

### CROSS-REFERENCE TO RELATED APPLICATIONS

This application claims the benefit under 35 U.S.C. § 119(e) of U.S. Provisional Patent Application Ser. No. 62/768,741, filed Nov. 16, 2018, entitled “FEEDBACK OF NEUROMUSCULAR ACTIVATION USING AUGMENTED REALITY,” the entire contents of which is incorporated by reference herein.

### FIELD OF THE INVENTION

The present technology relates to systems and methods that detect and interpret neuromuscular signals for use in performing actions in an augmented reality (AR) environment as well as other types of extended reality (XR) environments, such as a virtual reality (VR) environment, a mixed reality (MR) environment, and the like.

### BACKGROUND

AR systems provide users with an interactive experience of a real-world environment supplemented with virtual information by overlaying computer-generated perceptual or virtual information on aspects of the real-world environment. Physical objects in the real-world environment may be annotated with visual indicators within an AR environment generated by the AR system. The visual indicators may provide a user of the AR system with information about the physical objects.

In some computer applications that generate musculoskeletal representations of a human body for use in an AR environment, it may be desirable for the applications to know spatial positioning, orientation, and/or movement of one or more portion(s) of a user's body to provide a realistic and accurate representation of body movement and/or body position. Many of such musculoskeletal representations that reflect positioning, orientation, and/or movement of a user suffer from drawbacks, including flawed detection and feedback mechanisms, inaccurate outputs, lagging detection and output schemes, and other related issues.

### SUMMARY

According to aspects of the present technology, a computerized system for providing feedback to a user based on neuromuscular signals sensed from the user is described. The system may comprise a plurality of neuromuscular sensors and at least one computer processor. The plurality of neuromuscular sensors, which may be configured to sense a plurality of neuromuscular signals from the user, are arranged on one or more wearable devices. The least one computer processor may be programmed to: process the plurality of neuromuscular signals using one or more inference or statistical models; and provide feedback to the user based on one or both of: the processed plurality of neuromuscular signals, and information derived from the processed plurality of neuromuscular signals. The feedback may comprise visual feedback that includes information relating to one or both of: a timing of an activation of at least one motor unit of the user, and an intensity of the activation of the at least one motor unit of the user.

In an aspect, the feedback may comprise auditory feedback, or haptic feedback, or both auditory feedback and haptic feedback. The auditory feedback and the haptic feedback may relate to one or both of: the timing of the activation of the at least one motor unit of the user, and the intensity of the activation of the at least one motor unit of the user.

In another aspect, the visual feedback may further include a visualization relating to one or both of: the timing of the activation of the at least one motor unit of the user, and the intensity of the activation of the at least one motor unit of the user. The visualization may be provided within an augmented reality (AR) environment generated by an AR system or a virtual reality (VR) environment generated by a VR system. The visualization may depict at least one body part, with the at least one body part comprising any one or any combination of: a forearm of the user, a wrist of the user, and a leg of the user.

In a variation of this aspect, the at least one computer processor may be programmed to provide to the user a visualization of at least one target neuromuscular activity state. The at least one target neuromuscular activity state may be associated with performing a particular task.

In another variation of this aspect, the at least one computer processor may be programmed to determine, based on one or both of: the plurality of neuromuscular signals and information derived from the plurality of neuromuscular signals, deviation information from the at least one target neuromuscular activity state. The feedback provided to the user may comprise feedback based on the deviation information.

In yet another variation of this aspect, the at least one computer processor may be programmed to calculate a measure of muscle fatigue from one or both of: the plurality of neuromuscular signals and information derived from the plurality of neuromuscular signals. The visual feedback provided to the user may comprise a visual indication of the measure of muscle fatigue.

In an aspect, the at least one computer processor may be programmed to predict an outcome of a task or activity performed by the user based, at least in part, on one or both of: the plurality of neuromuscular signals and information derived from the plurality of neuromuscular signals. The feedback may comprise an indication of the predicted outcome.

In a variation of this aspect, the task or activity may be associated with an athletic movement or a therapeutic movement.

As will be appreciated, the present technology may encompass methods performed by or utilizing the systems of these aspects, and may further encompass computer-readable storage media storing code for the methods.

According to aspects of the present technology, a computerized system for providing feedback to a user based on neuromuscular signals sensed from the user is described. The system may comprise a plurality of neuromuscular sensors and at least one computer processor. The plurality of neuromuscular sensors, which may be configured to sense a plurality of neuromuscular signals from the user, may be arranged on one or more wearable devices. The at least one computer processor may be programmed to: process the plurality of neuromuscular signals using one or more inference or statistical models; and provide feedback to the user based on the processed plurality of neuromuscular signals. The feedback may be associated with one or more neuromuscular activity states of the user. The plurality of neuromuscular signals may relate to an athletic movement or a therapeutic movement performed by the user.

In an aspect, the feedback may comprise any one or any combination of: audio feedback, visual feedback, and haptic feedback.

In another aspect, the feedback may comprise visual feedback within an augmented reality (AR) environment generated by an AR system or a virtual reality (VR) environment generated by a VR system.

In a variation of this aspect, the visual feedback may comprise a visualization of one or both of: a timing of an activation of at least one motor unit of the user, and an intensity of the activation of the at least one motor unit of the user. The visualization may depict at least one body part, with the at least one body part comprising any one or any combination of: a forearm of the user, a wrist of the user, and a leg of the user. For example, the visualization may comprise a virtual representation or an augmented representation of the body part of the user, and the virtual representation or the augmented representation may depict the body part of the user acting with a greater activation force or moving with a larger degree of rotation than a reality-based activation force or a reality-based degree of rotation of the body party of the user.

In another variation of this aspect, the at least one computer processor may be programmed to provide to the user a visualization of at least one target neuromuscular activity state. The at least one target neuromuscular activity state may be associated with performing the athletic movement or the therapeutic movement. The visualization may comprise a virtual representation or an augmented representation of a body part of the user, and the virtual representation or the augmented representation may depict the body part of the user acting with a greater activation force or moving with a larger degree of rotation than a reality-based activation force or a reality-based degree of rotation of the body party of the user.

In variations of this aspect, the at least one computer processor may be programmed to determine, based on one or both of: the plurality of neuromuscular signals and information derived from the plurality of neuromuscular signals, deviation information from the at least one target neuromuscular activity state. The feedback may comprise a visualization based on the deviation information. In an implementation, the deviation information may be derived from a second plurality of neuromuscular signals processed by the at least one computer processor. In another implementation, the at least one computer processor may be programmed to predict an outcome of the athletic movement or the therapeutic movement performed by the user based, at least in part, on the deviation information, and the feedback may comprise an indication of the predicted outcome.

As will be appreciated, the present technology may encompass methods performed by or utilizing the systems of these aspects, and may further encompass computer-readable storage media storing code for the methods.

For example, according to an aspect of the present technology, a method for providing feedback to a user based on neuromuscular signals sensed from the user is described. The method, which may be performed by a computerized system, may comprise: receiving a plurality of neuromuscular signals sensed from the user using a plurality of neuromuscular sensors arranged on one or more wearable devices worn by the user; processing the plurality of neuromuscular signals using one or more inference or statistical models; and providing feedback to the user based on one or both of: the processed neuromuscular signals and information derived from the recorded neuromuscular signals. The feedback may comprise visual feedback that includes information relating to one or both of: a timing of an activation of at least one motor unit of the user, and an intensity of the activation of the at least one motor unit of the user.

In a variation of this aspect, the visual feedback may be provided within an augmented reality (AR) environment generated by an AR system or a virtual reality (VR) environment generated by a VR system.

In another variation of this aspect, the feedback may comprises auditory feedback or haptic feedback or auditory feedback and haptic feedback that relates to one or both of: the timing of the activation of the at least one motor unit of the user, and the intensity of the activation of the at least one motor unit of the user.

According to aspects of the present technology, a computerized system for providing feedback to a user based on neuromuscular signals sensed from the user is described. The system may comprise a plurality of neuromuscular sensors and at least one computer processor. The plurality of neuromuscular sensors, which may be configured to sense a plurality of neuromuscular signals from the user, may be arranged on one or more wearable devices. The at least one computer processor may be programmed to provide feedback to the user associated with one or both of: a timing of one or both of: a motor-unit activation and a muscle activation of the user, and an intensity of one or both of: the motor-unit activation and the muscle activation of the user. The feedback may be based on one or both of: the plurality of neuromuscular signals, and information derived from the plurality of neuromuscular signals.

In an aspect, the feedback may comprise audio feedback, or haptic feedback, or audio feedback and haptic feedback.

In another aspect, the feedback may comprise visual feedback.

In a variation of this aspect, the visual feedback may be provided within an augmented reality (AR) environment generated by an AR system or a virtual reality (VR) environment generated by a VR system. In one implementation, the feedback may comprise an instruction to the AR system to project, within the AR environment, a visualization of the timing or the intensity or the timing and the intensity on one or more body parts of the user. In another implementation, the feedback may comprise an instruction to the VR system to display, within the VR environment, a visualization of the timing or the intensity or the timing and the intensity on a virtual representation of one or more body parts of the user.

In an aspect, the at least one computer processor may be programmed to predict an outcome of a task based, at least in part, on one or both of: the plurality of neuromuscular signals and information derived from the plurality of neuromuscular signals. The feedback may comprise an indication of the predicted outcome.

In an aspect, the feedback may be provided during sensing of the plurality of neuromuscular signals.

In another aspect, the feedback may be provided in real-time.

In variations of this aspect, the plurality of neuromuscular signals may be sensed as the user is performing a particular task, and the feedback may be provided before the user completes performing the particular task. The particular task may be associated with an athletic movement or a therapeutic movement. For example, the therapeutic movement may be associated with monitoring a recovery associated with an injury. In another example, the feedback may be based, at least in part, on ergonomics associated with performing the particular task.

In an aspect, the at least one computer processor may be programmed to store one or both of: the plurality of neuromuscular signals and the information derived from the plurality of neuromuscular signals. The feedback may be based on one or both of: the stored plurality of neuromuscular signals and the stored information derived from the plurality of neuromuscular signals.

In a variation of this aspect, the feedback may be provided at a time when the plurality of neuromuscular signals are not being sensed.

In another aspect, the at least one computer processor may be programmed to provide to the user a visualization of a target neuromuscular activity associated with performing a particular task.

In a variation of this aspect, the target neuromuscular activity may comprise one or both of: a target timing of motor-unit activation or muscle activation or motor-unit and muscle activation of the user, and a target intensity of motor-unit activation or muscle activation or motor-unit and muscle activation of the user.

In another variation of this aspect, the visualization of the target neuromuscular activity may comprise a projection of the target neuromuscular activity onto one or more body parts of the user in an augmented reality (AR) environment generated by an AR system.

In yet another variation of this aspect, the visualization of the target neuromuscular activity may comprise an instruction to a virtual reality (VR) system to display a visualization of a timing of motor-unit activation or muscle activation or motor-unit and muscle activation of the user, or an intensity of the motor-unit activation or the muscle activation or the motor-unit activation and the muscle activation of the user, or both the timing and the intensity of the motor-unit activation or the muscle activation or the motor-unit action and the muscle activation of the user, within a VR environment generated by the VR system.

In a variation of this aspect, the at least one computer processor may be programmed to determine, based on one or both of: the plurality of neuromuscular signals and information derived from the plurality of neuromuscular signals, deviation information from the target neuromuscular activity. The feedback may comprise feedback based on the deviation information. In an implementation, the feedback based on the deviation information may comprise a visualization of the deviation information. For example, the visualization of the deviation information may comprise a projection of the deviation information onto one or more body parts of the user in an augmented reality (AR) environment generated by an AR system. In another example, the visualization of the deviation information may comprise an instruction provided to a virtual reality (VR) system to display the visualization of the deviation information on a virtual representation of one or more body parts of the user within a VR environment generated by the VR reality system. In yet another implementation, the at least one computer processor may be programmed to predict an outcome of a task based, at least in part, on the deviation information, and the feedback based on the deviation information may comprise an indication of the predicted outcome.

In an aspect, the at least one computer processor may be programmed to generate the target neuromuscular activity for the user based, at least in part, on one or both of: neuromuscular signals and information derived from the neuromuscular signals sensed during one or more performances of the particular task by the user or by a different user.

In a variation of this aspect, the at least one computer processor may be programmed to determine, based on one or more criteria, for each of the one or more performances of the particular task by the user or by the different user, a degree to which the particular task was performed well. The target neuromuscular activity may be generated for the user based on the degree to which each of the one or more performances of the particular task was performed well. The one or more criteria may include an indication from the user or from the different user about the degree to which the particular task was performed well.

In another variation of this aspect, the at least one computer processor may be programmed to determine, based on one or more criteria, for each of the one or more performances of the particular task by the user or by a different user, a degree to which the particular task was performed poorly. The target neuromuscular activity may be generated for the user based on the degree to which each of the one or more performances of the particular task was performed poorly. The one or more criteria may include an indication from the user or the different user about the degree to which the particular task was performed poorly.

In another aspect, the at least one computer processor may be programmed to calculate a measure of muscle fatigue from one or both of: the plurality of neuromuscular signals and information derived from the plurality of neuromuscular signals. The feedback may comprise an indication of the measure of muscle fatigue.

In a variation of this aspect, a calculation of the measure of muscle fatigue by the at least one computer processor may comprise determining spectral changes in the plurality of neuromuscular signals.

In another variation of this aspect, the indication of the measure of muscle fatigue may comprise a projection of the indication of the measure of muscle fatigue onto one or more body parts of the user in an augmented reality (AR) environment generated by an AR system.

In another variation of this aspect, the indication of the measure of muscle fatigue may comprise an instruction provided to a virtual reality (VR) system to display the indication of the measure of muscle fatigue within a VR environment generated by the VR system.

In another variation of this aspect, the at least one computer processor may be programmed to determine, based at least in part on the measure of muscle fatigue, an instruction to provide to the user to change a behavior of the user. The feedback may comprise an instruction to the user.

In another variation of this aspect, the at least one computer processor may be programmed to determine, based on the measure of muscle fatigue, whether a level of fatigue of the user is greater than a threshold level of muscle fatigue. The indication of the measure of muscle fatigue may comprise an alert about the level of fatigue, if the level of fatigue is determined to be greater than the threshold level of muscle fatigue.

In an aspect, the plurality of neuromuscular sensors may include at least one inertial measurement unit (IMU) sensor. The plurality of neuromuscular signals may comprise at least one neuromuscular signal sensed by the at least one IMU sensor.

In another aspect, the system may further comprise at least one auxiliary sensor configured to sense position information for one or more body parts of the user. The feedback may be based on the position information.

In a variation of this aspect, the at least one auxiliary sensor may comprise at least one camera.

In an aspect, the feedback provided to the user may comprise information associated with a performance of a physical task by the user.

In a variation of this aspect, the information associated with the performance of the physical task may comprise an indication of whether a force applied to a physical object during performance of the physical task was greater than a threshold force.

In another variation of this aspect, the information associated with the performance of the physical task may be provided to the user before the performance of the physical task is completed.

As will be appreciated, the present technology may encompass methods performed by or utilizing the systems of these aspects, and may further encompass computer-readable storage media storing code for the methods.

For example, according to an aspect of the present technology, a method for providing feedback for providing feedback to a user based on neuromuscular signals sensed from the user is described. The method, which may be performed by a computerized system, may comprise: using a plurality of neuromuscular sensors arranged on one or more wearable devices to sense a plurality of neuromuscular signals from the user; and providing feedback to the user associated with one or both of: a timing of a motor-unit activation of the user or a muscle activation of the user or both the motor-unit activation and the muscle activation of the user, and an intensity of a motor-unit activation of the user or a muscle activation of the user or both the motor-unit activation and the muscle activation of the user. The feedback may be based on one or both of: the sensed neuromuscular signals and information derived from the sensed neuromuscular signals.

In another example, according to an aspect of the present technology, a non-transitory computer-readable storage medium storing a program code for this method is described. That is, the program code, when executed by a computer, causes the computer to perform this method.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale.

FIG. 1 is a schematic diagram of a computer-based system for processing neuromuscular sensor data, such as signals obtained from neuromuscular sensors, to generate a musculoskeletal representation, in accordance with some embodiments of the technology described herein;

FIG. 2 is a schematic diagram of a distributed computer-based system that integrates an AR system with a neuromuscular activity system, in accordance with some embodiments of the technology described herein;

FIG. 3 shows a flowchart of a process for using neuromuscular signals to provide feedback to a user, in accordance with some embodiments of the technology described herein;

FIG. 4 shows a flowchart of a process for using neuromuscular signals to determine intensity, timing, and/or muscle activation, in accordance with some embodiments of the technology described herein;

FIG. 5 shows a flowchart of a process for using neuromuscular signals to provide a projected visualization feedback in an AR environment, in accordance with some embodiments of the technology described herein;

FIG. 6 shows a flowchart of a process for using neuromuscular signals to provide current and target musculoskeletal representations in an AR environment, in accordance with some embodiments of the technology described herein;

FIG. 7 shows a flowchart of a process for using neuromuscular signals to determine deviations from a target musculoskeletal representation, and to provide feedback to a user, in accordance with some embodiments of the technology described herein;

FIG. 8 shows a flowchart of a process for using neuromuscular signals to obtain target neuromuscular activity, in accordance with some embodiments of the technology described herein;

FIG. 9 shows a flowchart of a process for using neuromuscular activity to assess one or more task(s) and to provide feedback, in accordance with some embodiments of the technology described herein;

FIG. 10 shows a flowchart of a process for using neuromuscular signals to monitor muscle fatigue, in accordance with some embodiments of the technology described herein;

FIG. 11 shows a flowchart of a process for providing data to a trained inference model to obtain musculoskeletal information, in accordance with some embodiments of the technology described herein;

FIGS. 12A, 12B, 12C, and 12D schematically illustrate patch type wearable systems with sensor electronics incorporated thereon, in accordance with some embodiments of the technology described herein;

FIG. 13 illustrates a wristband having EMG sensors arranged circumferentially thereon, in accordance with some embodiments of the technology described herein;

FIG. 14A illustrates a wearable system with sixteen EMG sensors arranged circumferentially around a band configured to be worn around a user's lower arm or wrist, in accordance with some embodiments of the technology described herein;

FIG. 14B is a cross-sectional view through one of the sixteen EMG sensors illustrated in FIG. 14A;

FIG. 15 schematically illustrates a computer-based system that includes a wearable portion and a dongle portion, in accordance with some embodiments of the technology described herein;

FIG. 16 shows an example of an XR implementation in which feedback about a user may be provided to the user via an XR headset; and

FIG. 17 shows an example of an XR implementation in which feedback about a user may be provided to another person assisting the user.

### DETAILED DESCRIPTION

It is appreciated that there may be difficulty observing, describing, and communicating about neuromuscular activity, such as that performed by a person by moving one or more body part(s), such as an arm, a hand, a leg, a foot, etc. In particular, it may be difficult to process a timing and/or an intensity of motor-unit activations and muscle activations in such body part(s) in order to provide feedback to a person who performed or is performing certain movements of his or her body part(s). Skilled motor acts to be performed by humans may require precise coordinated activations of motor units and muscles, and learning such skilled acts may be hindered by difficulties in observing and communicating about motor-unit activations and muscle activations. Difficulty communicating about these activations can also be a hindrance to coaches, trainers (both human and automated/semi-automated ones), medical providers, and others who instruct humans to perform certain acts in athletics, performing arts, rehabilitation, and other areas. As will be appreciated, precise feedback regarding these activations is desirable for people learning to use neuromuscular control technology to control one or more system(s) (e.g., robotic systems, industrial control systems, gaming systems, AR systems, VR systems, other XR systems, etc.).

In some embodiments of the present technology described herein, systems and methods are provided for performing sensing and/or measurement(s) of neuromuscular signals, identification of activation of one or more neuromuscular structure(s), and delivering feedback to a user to provide information about the user's neuromuscular activation(s). In some embodiments, such feedback may be provided as any one or any combination of a visual display, an XR display (e.g., a MR, AR, and/or VR display), haptic feedback, an auditory signal, a user interface, and other types of feedback able to assist the user in performing certain movements or activities. Further, neuromuscular signal data may be combined with other data to provide more accurate feedback to the user. Such feedback to the user may take various forms, e.g., timing(s), intensity(ies), and/or muscle activation(s) relating to the neuromuscular activations of the user. Feedback may be delivered to the user instantaneously (e.g., in real-time or near real-time with minimal latency) or at some point in time after completing the movements or activities.

As will be appreciated, some systems of the present technology described herein may be used within an AR environment and/or a VR environment to provide such feedback to users. For instance, visualization of muscle and motor-unit activation(s) can be projected over a user's body within a display produced by an AR or VR system. Other feedback types, such as, for example, auditory tones or instructions, haptic buzzes, electrical feedback, etc., may be provided alone or in combination with visual feedback. Some embodiments of the present technology may provide a system that is capable of measuring or sensing a user's movement(s) through neuromuscular signals, comparing the movement(s) to a desired movement or movements, and providing feedback to the user about any differences or similarities between the desired movement(s) and the measured or sensed (i.e., actual) movement(s) of the user.

In some embodiments of the technology described herein, sensor signals may be used to predict information about a position and/or a movement of one or more portion(s) of a user's body (e.g., a leg, an arm, and/or a hand), which may be represented as a multi-segment articulated rigid-body system with joints connecting the multiple segments of the rigid-body system. For example, in the case of a hand movement, signals sensed by wearable neuromuscular sensors placed at locations on the user's body (e.g., the user's arm and/or wrist) may be provided as input to one or more inference model(s) trained to predict estimates of the position (e.g., absolute position, relative position, orientation) and the force(s) associated with a plurality of rigid segments in a computer-based musculoskeletal representation associated with a hand, for example, when the user performs one or more hand movements. The combination of position information and force information associated with segments of the musculoskeletal representation associated with a hand may be referred to herein as a “handstate” of the musculoskeletal representation. As a user performs different movements, a trained inference model may interpret neuromuscular signals sensed by the wearable neuromuscular sensors into position and force estimates (handstate information) that are used to update the musculoskeletal representation. Because the neuromuscular signals may be continuously sensed, the musculoskeletal representation may be updated in real-time, and a visual representation of one or more portion(s) of the user's body may be rendered (e.g., a hand within an AR or VR environment) based on current estimates of the handstate determined from the neuromuscular signals. As will be appreciated, an estimate of the user's handstate, determined using the user's neuromuscular signals, may be used to determine a gesture being performed by the user and/or to predict a gesture that the user will perform.

In some embodiments of the present technology, a system that senses neuromuscular signals may be coupled with a system that performs XR (e.g., AR or VR or MR) functions. For example, a system that senses neuromuscular signals used for determining a position of a body part (e.g., a hand, an arm, etc.) of a user may be used in conjunction with an AR system, such that the combined system may provide an improved AR experience for the user. Information gained by these systems may be used to improve the overall AR experience for the user. In one implementation, a camera included in the AR system may capture data that is used to improve an accuracy of a model of a musculoskeletal representation and/or to calibrate the model. Further, in another implementation, muscle activation data obtained by the system via sensed neuromuscular signals may be used to generate a visualization that may be displayed to the user in an AR environment. In yet another implementation, information displayed in the AR environment may be used as feedback to the user to permit the user to more accurately perform, e.g., gestures, or poses, or movements, etc., used for musculoskeletal input to the combined system. Further, control features may be provided in the combined system, which may permit predetermined neuromuscular activity to control aspects of the AR system.

In some embodiments of the present technology, musculoskeletal representations (e.g., handstate renderings) may include different types of representations that model user activity at different levels. For instance, such representations may include any one or any combination of an actual visual representation of a biomimetic (realistic) hand, a synthetic (robotic) hand, a low-dimensional embedded-space representation (e.g., by utilizing Principal Component Analysis (PCA), Isomaps, Local Linear Embedding (LLE), Sensible PCA, and/or another suitable technique to produce a low-dimensional representation), as well as an “internal representation” that may serve as input information for a gesture-based control operation (e.g., to control one or more function(s) of another application or another system, etc.). That is, in some implementations, hand-position information and/or force information may be provided as inputs for downstream algorithms but need not be directly rendered. As mentioned above, data captured by a camera may be used to assist in creating actual visual representations (e.g., improving an XR version of the user's hand using a hand image captured by the camera).

As discussed above, it may be beneficial to measure (e.g., sense and analyze) neuromuscular signals, to determine identifications of an activation of one or more neuromuscular structure(s), and to deliver feedback to the user to provide information about the user's neuromuscular activations. In some embodiments of the technology described herein, in order to obtain a reference for determining human movement, a system may be provided for measuring and modeling a human musculoskeletal system. All or portions of the human musculoskeletal system can be modeled as a multi-segment articulated rigid body system, with joints forming the interfaces between the different segments and joint angles defining the spatial relationships between connected segments in the model.

Constraints on the movement at a joint are governed by the type of joint connecting the segments and the biological structures (e.g., muscles, tendons, ligaments) that may restrict the range of movement at the joint. For example, the shoulder joint connecting the upper arm to a torso or a human subject, and a hip joint connecting an upper leg to the torso, are ball and socket joints that permit extension and flexion movements as well as rotational movements. By contrast, an elbow joint connecting the upper arm and a lower arm (or forearm), and a knee joint connecting the upper leg and a lower leg of the human subject, allow for a more limited range of motion. In this example, a multi-segment articulated rigid body system may be used to model portions of the human musculoskeletal system. However, it should be appreciated that although some segments of the human musculoskeletal system (e.g., the forearm) may be approximated as a rigid body in the articulated rigid body system, such segments may each include multiple rigid structures (e.g., the forearm may include ulna and radius bones), which may enable more complex movements within the segment that is not explicitly considered by the rigid body model. Accordingly, a model of an articulated rigid body system for use with some embodiments of the technology described herein may include segments that represent a combination of body parts that are not strictly rigid bodies. It will be appreciated that physical models other than the multi-segment articulated rigid body system may be used to model portions of the human musculoskeletal system without departing from the scope of this disclosure.

Continuing with the example above, in kinematics, rigid bodies are objects that exhibit various attributes of motion (e.g., position, orientation, angular velocity, acceleration). Knowing the motion attributes of one segment of the rigid body enables the motion attributes for other segments of the rigid body to be determined based on constraints in how the segments are connected. For example, the hand may be modeled as a multi-segment articulated body, with the joints in the wrist and each finger forming interfaces between the multiple segments in the model. Movements of the segments in the rigid body model can be simulated as an articulated rigid body system in which position (e.g., actual position, relative position, or orientation) information of a segment relative to other segments in the model are predicted using a trained inference model, as described in more detail below.

For some embodiments of the present technology, the portion of the human body approximated by a musculoskeletal representation may be a hand or a combination of a hand with one or more arm segments. The information used to describe a current state of the positional relationships between segments, force relationships for individual segments or combinations of segments, and muscle and motor-unit activation relationships between segments in the musculoskeletal representation is referred to herein as the “handstate” of the musculoskeletal representation (see other discussions of handstate herein). It should be appreciated, however, that the techniques described herein are also applicable to musculoskeletal representations of portions of the body other than the hand, including, hut not limited to, an arm, a leg, a foot, a torso, a neck, or any combination of the foregoing.

In addition to spatial (e.g., position and/or orientation) information, some embodiments of the present technology enable a prediction of force information associated with one or more segments of the musculoskeletal representation. For example, linear forces or rotational (torque) forces exerted by one or more segments may be estimated. Examples of linear forces include, but are not limited to, the force of a finger or hand pressing on a solid object such as a table, and a force exerted when two segments (e.g., two fingers) are pinched together. Examples of rotational forces include, but are not limited to, rotational forces created when a segment, such as in a wrist or a finger, is twisted or flexed relative to another segment. In some embodiments, the force information determined as a portion of a current handstate estimate includes one or more of: pinching force information, grasping force information, and information about co-contraction forces between muscles represented by the musculoskeletal representation. It should be appreciated that there may be multiple forces associated with a segment of a musculoskeletal representation. For example, there are multiple muscles in a forearm segment, and force acting on the forearm segment may be predicted based on an individual muscle or based on one or more group(s) of muscles (e.g., flexors, extensors, etc.).

As used herein, the term “gestures” may refer to a static or dynamic configuration of one or more body parts including a position of the one or more body parts and forces associated with the configuration. For example, gestures may include discrete gestures, such as placing or pressing the palm of a hand down on a solid surface, or grasping a ball, or pinching two fingers together (e.g., to form a pose); or continuous gestures, such as waving a finger back and forth, grasping and throwing a ball, rotating a wrist in a direction; or a combination of discrete and continuous gestures. Gestures may include covert gestures that may be imperceptible to another person, such as slightly tensing a joint by co-contracting opposing muscles or using sub-muscular activations. In training an inference model, gestures may be defined using an application configured to prompt a user to perform the gestures or, alternatively, gestures may be arbitrarily defined by a user. The gestures performed by the user may include symbolic gestures (e.g., gestures mapped to other gestures, interactions, or commands, for example, based on a gesture vocabulary that specifies the mapping). In some cases, hand and arm gestures may be symbolic and used to communicate according to cultural standards.

In accordance with some embodiments of the technology described herein, signals sensed by one or more wearable sensor(s) may be used to control an XR system. The inventors have discovered that a number of muscular activation states of a user may be identified from such sensed signals and/or from information based on or derived from such sensed signals to enable improved control of the XR system. Neuromuscular signals may be used directly as an input to an XR system (e.g. by using motor-unit action potentials as an input signal) and/or the neuromuscular signals may be processed (including by using an inference model as described herein) for the purpose of determining a movement, a force, and/or a position of a part of the user's body (e.g. fingers, hand, wrist, leg, etc.). Various operations of the XR system may be controlled based on identified muscular activation states. An operation of the XR system may include any aspect of the XR system that the user can control based on sensed signals from the wearable sensor(s). The muscular activation states may include, but are not limited to, a static gesture or pose performed by the user, a dynamic gesture or motion performed by the user, a sub-muscular activation state of the user, a muscular tensing or relaxation performed by the user, or any combination of the foregoing. For instance, control of the XR system may include control based on activation of one or more individual motor units, e.g., control based on a detected sub-muscular activation state of the user, such as a sensed tensing of a muscle. Identification of one or more muscular activation state(s) may allow a layered or multi-level approach to controlling operation(s) of the XR system. For instance, at a first layer/level, one muscular activation state may indicate that a mode of the XR system is to be switched from a first mode (e.g., an XR interaction mode) to a second mode (e.g., a control mode for controlling operations of the XR system); at a second layer/level, another muscular activation state may indicate an operation of the XR system that is to be controlled; and at a third layer/level, yet another muscular activation state may indicate how the indicated operation of the XR system is to be controlled. It will be appreciated that any number of muscular activation states and layers may be used without departing from the scope of this disclosure. For example, in some embodiments, one or more muscular activation state(s) may correspond to a concurrent gesture based on activation of one or more motor units, e.g., the user's hand bending at the wrist while pointing the index finger. In some embodiments, one or more muscular activation state(s) may correspond to a sequence of gestures based on activation of one or more motor units, e.g., the user's hand bending at the wrist upwards and then downwards. In some embodiments, a single muscular activation state may both indicate to switch into a control mode and indicate the operation of the XR system that is to be controlled. As will be appreciated, the phrases “sensed and recorded”, “sensed and collected”, “recorded”, “collected”, “obtained”, and the like, when used in conjunction with a sensor signal comprises a signal detected or sensed by the sensor. As will be appreciated, the signal may be sensed and recorded or collected without storage in a nonvolatile memory, or the signal may be sensed and recorded or collected with storage in a local nonvolatile memory or in an external nonvolatile memory. For example, after detection or being sensed, the signal may be stored at the sensor “as-detected” (i.e., raw), or the signal may undergo processing at the sensor prior to storage at the sensor, or the signal may be communicated (e.g., via a Bluetooth technology or the like) to an external device for processing and/or storage, or any combination of the foregoing.

According to some embodiments of the present technology, the muscular activation states may be identified, at least in part, from raw (e.g., unprocessed) sensor signals obtained (e.g., sensed) by one or more wearable sensor(s). In some embodiments, the muscular activation states may be identified, at least in part, from information based on the raw sensor signals (e.g., processed sensor signals), where the raw sensor signals obtained by the one or more wearable sensor(s) are processed to perform, e.g., amplification, filtering, rectification, and/or other form of signal processing, examples of which are described in more detail below. In some embodiments, the muscular activation states may be identified, at least in part, from an output of one or more trained inference model(s) that receive the sensor signals (raw or processed versions of the sensor signals) as input(s).

As noted above, muscular activation states, as determined based on sensor signals in accordance with one or more of the techniques described herein, may be used to control various aspects and/or operations of an XR system. Such control may reduce the need to rely on cumbersome and inefficient input devices (e.g., keyboards, mouses, touchscreens, etc.). For example, sensor data (e.g., signals obtained from neuromuscular sensors or data derived from such signals) may be obtained and muscular activation states may be identified from the sensor data without the user having to carry a controller and/or other input device, and without having the user remember complicated button or key manipulation sequences. Also, the identification of the neuromuscular activation states (e.g., poses, gestures, varying degrees of force associated with the neuromuscular activation states, etc.) from the sensor data can be performed relatively fast, thereby reducing the response times and latency associated with controlling the XR system. Signals sensed by wearable sensors placed at locations on the user's body may be provided as input to an inference model trained to generate spatial and/or force information for rigid segments of a multi-segment articulated rigid-body model of a human body, as mentioned above. The spatial information may include, for example, position information of one or more segments, orientation information of one or more segments, joint angles between segments, and the like. Based on the input, and as a result of training, the inference model may implicitly represent inferred motion of the articulated rigid body under defined movement constraints. The trained inference model may output data useable for applications such as applications for rendering a representation of the user's body in an XR environment, in which the user may interact with physical and/or virtual objects, and/or applications for monitoring the user's movements as the user performs a physical activity to assess, for example, whether the user is performing the physical activity in a desired manner. As will be appreciated, the output data from the trained inference model may be used for applications other than those specifically identified herein. For instance, movement data obtained by a single movement sensor positioned on the user (e.g., on the user's wrist or arm) may be provided as input data to a trained inference model. Corresponding output data generated by the trained inference model may be used to determine spatial information for one or more segments of a multi-segment articulated rigid body model for the user. For example, the output data may be used to determine the position and/or the orientation of one or more segments in the multi-segment articulated rigid body model. In another example, the output data may be used to determine angles between connected segments in the multi-segment articulated rigid-body model.

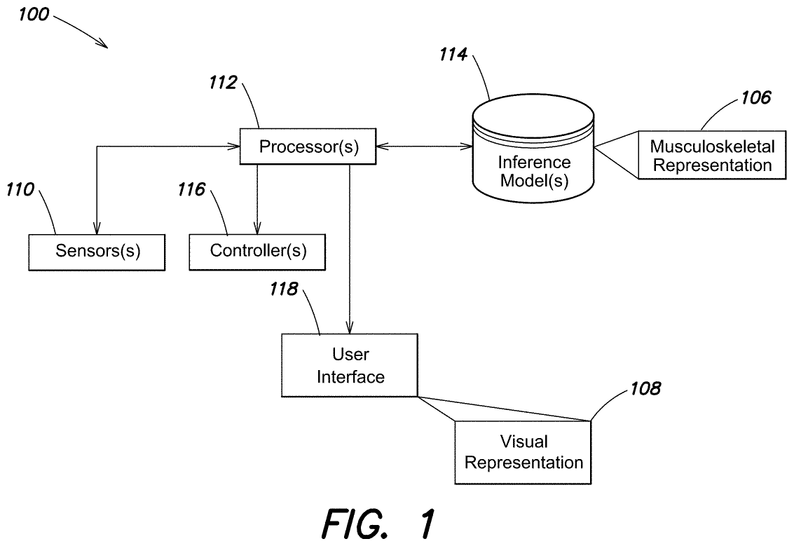

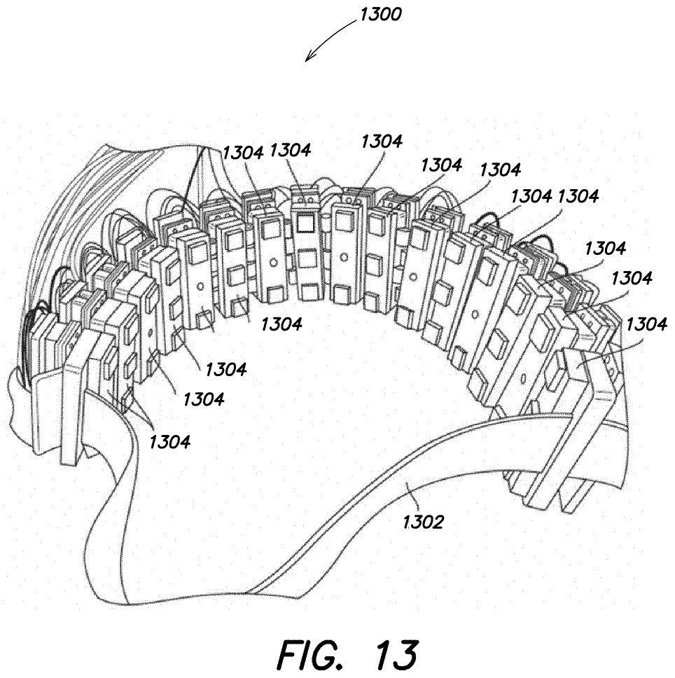

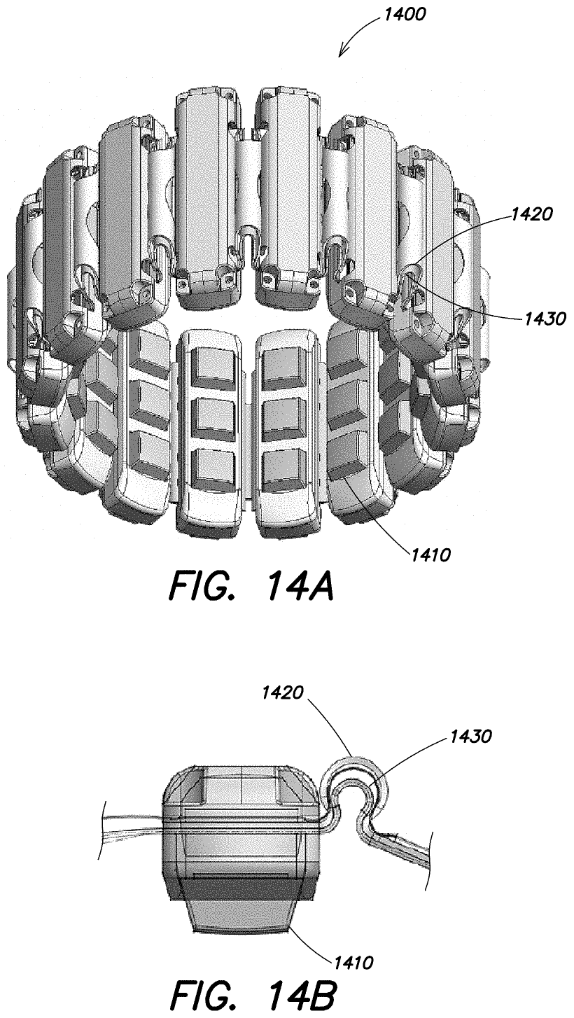

Turning now to the figures, FIG. 1 schematically illustrates a system 100, for example, a neuromuscular activity system, in accordance with some embodiments of the technology described herein. The system may comprise one or more sensor(s) 110 configured to sense (e.g., detect, measure, and/or record) signals resulting from activation of motor units within one or more portion(s) of a human body. Such activation may involve a visible movement of the portion(s) of the human body, or a movement that may not be readily seen with a naked eye. The sensor(s) 110 may include one or more neuromuscular sensor(s) configured to sense signals arising from neuromuscular activity in skeletal muscle of a human body (e.g., carried on a wearable device) without requiring the use of auxiliary devices (e.g., cameras, global positioning systems, laser scanning systems) and also without requiring the use of an external sensor or device (i.e., not carried on the wearable device), as discussed below with reference to FIGS. 13 and 14A. As will be appreciated, although not required, one or more auxiliary device(s) may be used in conjunction with the neuromuscular sensor(s).

The term “neuromuscular activity” as used herein refers to neural activation of spinal motor neurons or units that innervate a muscle, muscle activation, muscle contraction, or any combination of the neural activation, muscle activation, and muscle contraction. The one or more neuromuscular sensor(s) may include one or more electromyography (EMG) sensors, one or more mechanomyography (MMG) sensors, one or more sonomyography (SMG) sensors, a combination of two or more types of EMG sensors, MMG sensors, and SMG sensors, and/or one or more sensors of any suitable type able to detect neuromuscular signals. In some embodiments of the present technology, information relating to an interaction of a user with a physical object in an XR environment (e.g., an AR, MR, and/or VR environment) may be determined from neuromuscular signals sensed by the one or more neuromuscular sensor(s). Spatial information (e.g., position and/or orientation information) and force information relating to the movement may be predicted based on the sensed neuromuscular signals as the user moves over time. In some embodiments, the one or more neuromuscular sensor(s) may sense muscular activity related to movement caused by external objects, for example, movement of a hand being pushed by an external object.

The term “neuromuscular activity state” or “neuromuscular activation state” may comprise any information relating to one or more characteristics of a neuromuscular activity, including but not limited to: a strength of a muscular or sub-muscular contraction, an amount of force exerted by a muscular or sub-muscular contraction, a performance of a pose or a gesture and/or any varying amount of force(s) associated with that performance, spatio-temporal positioning of one or more body parts or segments, a combination of position information and force information associated with segments of a musculoskeletal representation associated with a hand (e.g., handstate) or other body part, any pattern by which muscles become active and/or increase their firing rate, and angles between connected segments in a multi-segment articulated rigid-body model. Accordingly, the term “neuromuscular activity state” or “neuromuscular activation state” is meant to encompass any information relating to sensed, detected, and/or recorded neuromuscular signals and/or information derived from those neuromuscular signals.

The one or more sensor(s) 110 may include one or more auxiliary sensor(s), such as one or more photoplethysmography (PPG) sensors, which detect vascular changes (e.g., changes in blood volume) and/or one or more Inertial Measurement Unit(s) or IMU(s), which measure a combination of physical aspects of motion, using, for example, an accelerometer, a gyroscope, a magnetometer, or any combination of one or more accelerometers, gyroscopes and magnetometers. In some embodiments, one or more IMU(s) may be used to sense information about movement of the part of the body on which the IMU(s) is or are attached, and information derived from the sensed IMU data (e.g., position and/or orientation information) may be tracked as the user moves over time. For example, one or more IMU(s) may be used to track movements of portions (e.g., arms, legs) of a user's body proximal to the user's torso relative to the IMU(s) as the user moves over time.

In embodiments that include at least one IMU and one or more neuromuscular sensor(s), the IMU(s) and the neuromuscular sensor(s) may be arranged to detect movement of different parts of a human body. For example, the IMU(s) may be arranged to detect movements of one or more body segments proximal to the torso (e.g., movements of an upper arm), whereas the neuromuscular sensors may be arranged to detect motor unit activity within one or more body segments distal to the torso (e.g., movements of a lower arm (forearm) or a wrist). It should be appreciated, however, that the sensors (i.e., the IMU(s) and the neuromuscular sensor(s)) may be arranged in any suitable way, and embodiments of the technology described herein are not limited based on the particular sensor arrangement. For example, in some embodiments, at least one IMU and a plurality of neuromuscular sensors may be co-located on a body segment to track motor unit activity and/or movements of the body segment using different types of measurements. In one implementation, an IMU and a plurality of EMG sensors may be arranged on a wearable device structured to be worn around the lower arm or the wrist of a user. In such an arrangement, the IMU may be configured to track, over time, movement information (e.g., positioning and/or orientation) associated with one or more arm segments, to determine, for example, whether the user has raised or lowered his/her arm, whereas the EMG sensors may be configured to determine finer-grained or more subtle movement information and/or sub-muscular information associated with activation of muscular or sub-muscular structures in muscles of the wrist and/or the hand.

As the tension of a muscle increases during performance of a motor task, the firing rates of active neurons increase, and additional neurons may become active, which is a process referred to as motor-unit recruitment. A motor unit is made up of a motor neuron and skeletal muscle fibers innervated by that motor neuron's axonal terminals. Groups of motor units often work together to coordinate a contraction of a single muscle; all of the motor units within a muscle are considered a motor pool.

The pattern by which neurons become active and increase their firing rate may be stereotyped, such that the expected motor unit recruitment patterns may define an activity manifold associated with standard or normal movement. In some embodiments, sensor signals may identify activation of a single motor unit or a group of motor units that are “off-manifold,” in that the pattern of motor-unit activation is different than an expected or typical motor-unit recruitment pattern. Such off-manifold activation may be referred to herein as, “sub-muscular activation” or “activation of a sub-muscular structure,” where a sub-muscular structure refers to the single motor unit or the group of motor units associated with the off-manifold activation. Examples of off-manifold motor-unit recruitment patterns include, but are not limited to, selectively activating a high-threshold motor unit without activating a lower-threshold motor unit that would normally be activated earlier in the recruitment order and modulating the firing rate of a motor unit across a substantial range without modulating the activity of other neurons that would normally be co-modulated in typical motor-unit recruitment patterns. The one or more neuromuscular sensor(s) may be arranged relative to the human body to sense sub-muscular activation without observable movement, i.e., without a corresponding movement of the body that can be readily observed by naked eyes. Sub-muscular activation may be used, at least in part, to provide information to an AR or VR system and/or to interact with a physical object in an AR or VR environment produced by the AR or VR system.

Some or all of the sensor(s) 110 may each include one or more sensing components configured to sense information about a user. In the case of IMUs, the sensing component(s) of an IMU may include one or more: accelerometer, gyroscope, magnetometer, or any combination thereof, to measure or sense characteristics of body motion, examples of which include, but are not limited to, acceleration, angular velocity, and a magnetic field around the body during the body motion. In the case of neuromuscular sensors, the sensing component(s) may include, but are not limited to, electrodes that detect electric potentials on the surface of the body (e.g., for EMG sensors), vibration sensors that measure skin surface vibrations (e.g., for MMG sensors), acoustic sensing components that measure ultrasound signals (e.g., for SMG sensors) arising from muscle activity, or any combination thereof. Optionally, the sensor(s) 110 may include any one or any combination of: a thermal sensor that measures the user's skin temperature (e.g., a thermistor); a cardio sensor that measure's the user's pulse, heart rate, a moisture sensor that measures the user's state of perspiration, and the like. Exemplary sensors that may be used as part of the one or more sensor(s) 110, in accordance with some embodiments of the technology disclosed herein, are described in more detail in U.S. Pat. No. 10,409,371 entitled “METHODS AND APPARATUS FOR INFERRING USER INTENT BASED ON NEUROMUSCULAR SIGNALS,” which is incorporated by reference herein in its entirety.

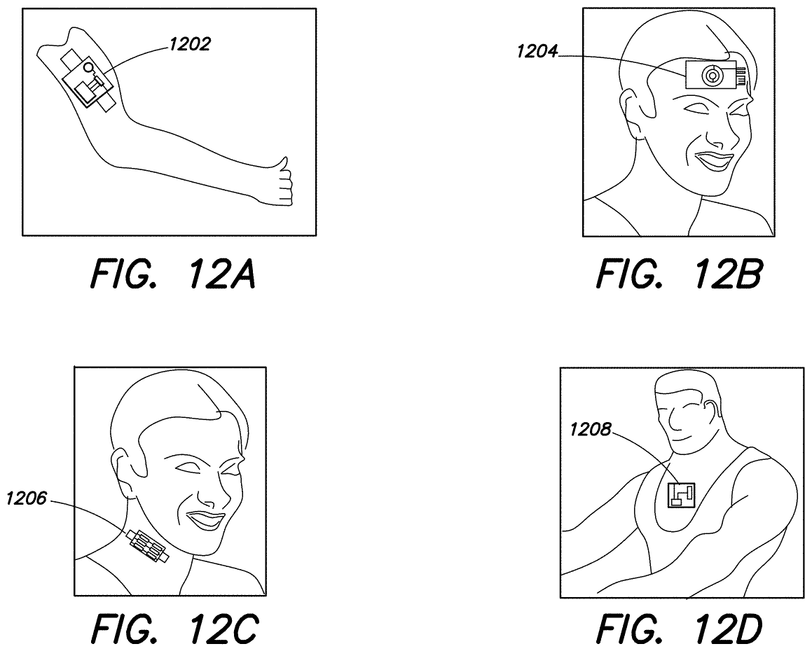

In some embodiments, the one or more sensor(s) 110 may comprise a plurality of sensors 110, and at least some of the plurality of sensors 110 may be arranged as a portion of a wearable device structured to be worn on or around part of a user's body. For example, in one non-limiting example, an IMU and a plurality of neuromuscular sensors are arranged circumferentially around an adjustable and/or elastic band, such as a wristband or an armband structured to be worn around a user's wrist or arm, as described in more detail below. In some embodiments, multiple wearable devices, each having one or more IMU(s) and/or one or more neuromuscular sensor(s) included thereon, may be used to determine information relating to an interaction of a user with a physical object based on activation from muscular and/or sub-muscular structures and/or based on movement that involves multiple parts of the body. Alternatively, at least some of the sensors 110 may be arranged on a wearable patch configured to be affixed to a portion of the user's body. FIGS. 12A-12D show various types of wearable patches. FIG. 12A shows a wearable patch 1202 in which circuitry for an electronic sensor may be printed on a flexible substrate that is structured to adhere to an arm, e.g., near a vein to sense blood flow in the user. The wearable patch 1202 may be an RFID-type patch, which may transmit sensed information wirelessly upon interrogation by an external device. FIG. 12B shows a wearable patch 1204 in which an electronic sensor may be incorporated on a substrate that is structured to be worn on the user's forehead, e.g., to measure moisture from perspiration. The wearable patch 1204 may include circuitry for wireless communication, or may include a connector structured to be connectable to a cable, e.g., a cable attached to a helmet, a heads-mounted display, or another external device. The wearable patch 1204 may be structured to adhere to the user's forehead or to be held against the user's forehead by, e.g., a headband, skullcap, or the like. FIG. 12C shows a wearable patch 1206 in which circuitry for an electronic sensor may be printed on a substrate that is structured to adhere to the user's neck, e.g., near the user's carotid artery to sense flood flow to the user's brain. The wearable patch 1206 may be an RFID-type patch or may include a connector structured to connect to external electronics. FIG. 12D shows a wearable patch 1208 in which an electronic sensor may be incorporated on a substrate that is structured to be worn near the user's heart, e.g., to measure the user's heartrate or to measure blood flow to/from the user's heart. As will be appreciated, wireless communication is not limited to RFID technology, and other communication technologies may be employed. Also, as will be appreciated, the sensors 110 may be incorporated on other types of wearable patches that may be structured differently from those shown in FIGS. 12A-12D.

In one implementation, the sensor(s) 110 may include sixteen neuromuscular sensors arranged circumferentially around a band (e.g., an adjustable strap, an elastic band, etc.) structured to be worn around a user's lower arm (e.g., encircling the user's forearm). For example, FIG. 13 shows an embodiment of a wearable system 1300 in which neuromuscular sensors 1304 (e.g., EMG sensors) are arranged circumferentially around a hand 1302. It should be appreciated that any suitable number of neuromuscular sensors may be used, and the number and arrangement of neuromuscular sensors used may depend on the particular application for which the wearable system is used. For example, a wearable armband or wristband may be used to generate control information for controlling an XR system, controlling a robot, controlling a vehicle, scrolling through text, controlling a virtual avatar, or any other suitable control task. In some embodiments, the band 1302 may also include one or more IMUs (not shown) for obtaining movement information, as discussed above.

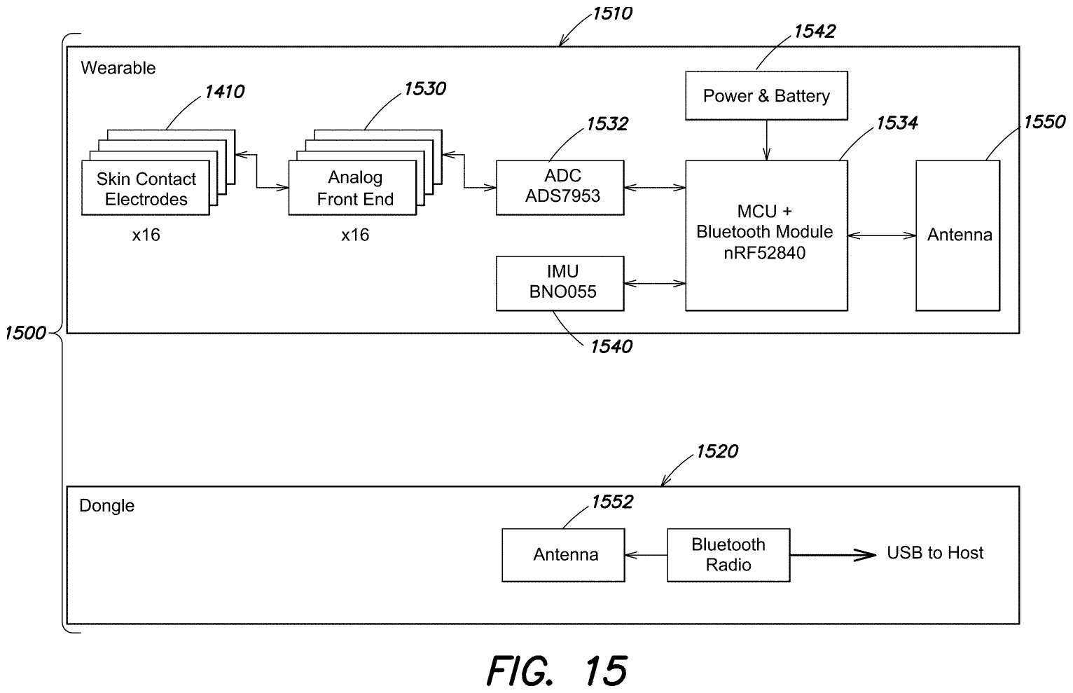

FIGS. 14A-14B and 15 show other embodiments of wearable systems of the present technology. In particular, FIG. 14A illustrates a wearable system 1400 with a plurality of sensors 1410 arranged circumferentially around an elastic band 1420 structured to be worn around a user's lower arm or wrist. The sensors 1410 may be neuromuscular sensors (e.g., EMG sensors). As shown, there may be sixteen sensors 1410 arranged circumferentially around the elastic band 1420 at a regular spacing. It should be appreciated that any suitable number of sensors 1410 may be used, and the spacing need not be regular. The number and arrangement of the sensors 1410 may depend on the particular application for which the wearable system is used. For instance, the number and arrangement of the sensors 1410 may differ when the wearable system is to be worn on a wrist in comparison with a thigh. A wearable system (e.g., armband, wristband, thighband, etc.) can be used to generate control information for controlling a robot, controlling a vehicle, scrolling through text, controlling a virtual avatar, and/or performing any other suitable control task.

In some embodiments, the sensors 1410 may include only a set of neuromuscular sensors (e.g., EMG sensors). In other embodiments, the sensors 1410 may include a set of neuromuscular sensors and at least one auxiliary device. The auxiliary device(s) may be configured to continuously sense and record one or a plurality of auxiliary signal(s). Examples of auxiliary devices include, but are not limited to, IMUs, microphones, imaging devices (e.g., cameras), radiation-based sensors for use with a radiation-generation device (e.g., a laser-scanning device), heart-rate monitors, and other types of devices, which may capture a user's condition or other characteristics of the user. As shown in FIG. 14A, the sensors 1410 may be coupled together using flexible electronics 1430 incorporated into the wearable system. FIG. 14B illustrates a cross-sectional view through one of the sensors 1410 of the wearable system 1400 shown in FIG. 14A.

In some embodiments, the output(s) of one or more sensing component(s) of the sensors 1410 can be processed using hardware signal-processing circuitry (e.g., to perform amplification, filtering, and/or rectification). In other embodiments, at least some signal processing of the output(s) of the sensing component(s) can be performed using software. Thus, signal processing of signals sensed or obtained by the sensors 1410 can be performed by hardware or by software, or by any suitable combination of hardware and software, as aspects of the technology described herein are not limited in this respect. A non-limiting example of a signal-processing procedure used to process data obtained by the sensors 1410 is discussed in more detail below in connection with FIG. 15.

FIGS. 15 illustrates a schematic diagram with internal components of a wearable system 1500 with sixteen sensors (e.g., EMG sensors), in accordance with some embodiments of the technology described herein. As shown, the wearable system includes a wearable portion 1510 and a dongle portion 1520. Although not illustrated, the dongle portion 1520 is in communication with the wearable portion 1510 (e.g., via Bluetooth or another suitable short range wireless communication technology). The wearable portion 1510 includes the sensors 1410, examples of which are described above in connection with FIGS. 14A and 14B. The sensors 1410 provide output (e.g., signals) to an analog front end 1530, which performs analog processing (e.g., noise reduction, filtering, etc.) on the signals. Processed analog signals produced by the analog front end 1530 are then provided to an analog-to-digital converter 1532, which converts the processed analog signals to digital signals that can be processed by one or more computer processors. An example of a computer processor that may be used in accordance with some embodiments is a microcontroller (MCU) 1534. The MCU 1534 may also receive inputs from other sensors (e.g., an IMU 1540) and from a power and battery module 1542. As will be appreciated, the MCU 1534 may receive data from other devices not specifically shown. A processing output by the MCU 1534 may be provided to an antenna 1550 for transmission to the dongle portion 1520.

The dongle portion 1520 includes an antenna 1552 that communicates with the antenna 1550 of the wearable portion 1510. Communication between the antennas 1550 and 1552 may occur using any suitable wireless technology and protocol, non-limiting examples of which include radiofrequency signaling and Bluetooth. As shown, the signals received by the antenna 1552 of the dongle portion 1520 may be provided to a host computer for further processing, for display, and/or for effecting control of a particular physical or virtual object or objects (e.g., to perform a control operation in an AR environment).

Although the examples provided with reference to FIGS. 14A, 14B, and 15 are discussed in the context of interfaces with EMG sensors, it is to be understood that the wearable systems described herein can also be implemented with other types of sensors, including, but not limited to, mechanomyography (MMG) sensors, sonomyography (SMG) sensors, and electrical impedance tomography (EIT) sensors.

Returning to FIG. 1, in some embodiments, sensor data or signals obtained by the sensor(s) 110 may be processed to compute additional derived measurements, which may then be provided as input to an inference model, as described in more detail below. For example, signals obtained from an IMU may be processed to derive an orientation signal that specifies the orientation of a segment of a rigid body over time. The sensor(s) 110 may implement signal processing using components integrated with the sensing components of the sensor(s) 110, or at least a portion of the signal processing may be performed by one or more components in communication with, but not directly integrated with the sensing components of the sensor(s) 110.

The system 100 also includes one or more computer processor(s) 112 programmed to communicate with the sensor(s) 110. For example, signals obtained by one or more of the sensor(s) 110 may be output from the sensor(s) 110 and provided to the processor(s) 112, which may be programmed to execute one or more machine learning algorithm(s) to process the signals output by the sensor(s) 110. The algorithm(s) may process the signals to train (or retrain) one or more inference model(s) 114, and the trained (or retrained) inference model(s) 114 may be stored for later use in generating a musculoskeletal representation. As will be appreciated, in some embodiments of the present technology, the inference model(s) 114 may include at least one statistical model. Non-limiting examples of inference models that may be used in accordance with some embodiments of the present technology to predict, e.g., handstate information based on signals from the sensor(s) 110 are discussed in U.S. patent application Ser. No. 15/659,504 filed Jul. 25, 2017, entitled “SYSTEM AND METHOD FOR MEASURING THE MOVEMENTS OF ARTICULATED RIGID BODIES,” which is incorporated by reference herein in its entirety. It should be appreciated that any type or combination of types of inference model(s) may be used, such as ones that are pre-trained, ones that are trained with user input, and/or ones that are periodically adapted or retrained based on further input.

Some inference models may have a long-standing focus on producing inferences achieved through building and fitting probability models to compute quantitative measures of confidence to determine relationships that are unlikely to result from noise or randomly. Machine-learning models may strive to produce predictions by identifying patterns, often in rich and unwieldy datasets. To some extent, robust machine-learning models may depend on datasets used during a training phase, which may be inherently related to data analysis and statistics. Accordingly, as used herein, the term “inference model” should be broadly construed to encompass inference models, machine-learning models, statistical models, and combinations thereof built to produce inferences, predictions, and/or otherwise used in the embodiments described herein.

In some embodiments of the present technology, the inference model(s) 114 may include a neural network and, for example, may be a recurrent neural network. In some embodiments, the recurrent neural network may be a long short-term memory (LSTM) neural network. It should be appreciated, however, that the recurrent neural network is not limited to be an LSTM neural network and may have any other suitable architecture. For example, in some embodiments, the recurrent neural network may be any one or any combination of: a fully recurrent neural network, a gated recurrent neural network, a recursive neural network, a Hopfield neural network, an associative memory neural network, an Elman neural network, a Jordan neural network, an echo state neural network, a second order recurrent neural network, and/or any other suitable type of recurrent neural network. In other embodiments, neural networks that are not recurrent neural networks may be used. For example, deep neural networks, convolutional neural networks, and/or feedforward neural networks, may be used.

In some embodiments of the present technology, the inference model(s) 114 may produce one or more discrete output(s). Discrete outputs (e.g., classification labels) may be used, for example, when a desired output is to know whether a particular pattern of activation (including individual biologically produced neural spiking events) is currently being performed by a user, as detected via neuromuscular signals obtained from the user. For example, the inference model(s) 114 may be trained to estimate whether the user is activating a particular motor unit, activating a particular motor unit at a particular timing, activating a particular motor unit with a particular firing pattern, and/or activating a particular combination of motor units. On a shorter timescale, a discrete classification may be output and used in some embodiments to estimate whether a particular motor unit fired an action potential within a given amount of time. In such a scenario, estimates from the inference model(s) 114 may then be accumulated to obtain an estimated firing rate for that motor unit.

In embodiments of the present technology in which an inference model is implemented as a neural network configured to output a discrete output (e.g., a discrete signal), the neural network may include a softmax layer, such that the outputs of the inference model add up to one and may be interpreted as probabilities. For instance, outputs of the softmax layer may be a set of values corresponding to a respective set of control signals, with each value indicating a probability that the user wants to perform a particular control action. As one non-limiting example, the outputs of the softmax layer may be a set of three probabilities (e.g., 0.92, 0.05, and 0.03) indicating the respective probabilities that a detected pattern of activity is one of three known patterns.

It should be appreciated that when an inference model is a neural network configured to output a discrete output (e.g., a discrete signal), the neural network is not required to produce outputs that add up to one. For example, instead of a softmax layer, the output layer of the neural network may be a sigmoid layer, which does not restrict the outputs to probabilities that add up to one. In such embodiments of the present technology, the neural network may be trained with a sigmoid cross-entropy cost. Such an implementation may be advantageous in cases where multiple different control actions may occur within a threshold amount of time and it is not important to distinguish an order in which these control actions occur (e.g., a user may activate two patterns of neural activity within the threshold amount of time). In some embodiments, any other suitable non-probabilistic multi-class classifier may be used, as aspects of the technology described herein arc not limited in this respect.

In some embodiments of the technology described herein, an output of the inference model(s) 114 may be a continuous signal rather than a discrete output (e.g., a discrete signal). For example, the model(s) 114 may output an estimate of a firing rate of each motor unit, or the model(s) 114 may output a time-series electrical signal corresponding to each motor unit or sub-muscular structure. Further, the model may output an estimate of a mean firing rate of all of the motor units within a designated functional group (e.g., within a muscle or a group of muscles).

It should be appreciated that aspects of the technology described herein are not limited to using neural networks, as other types of inference models may be employed in some embodiments. For example, in some embodiments, the inference model(s) 114 may comprise a Hidden Markov Model (HMM), a switching HMM in which switching allows for toggling among different dynamic systems, dynamic Bayesian networks, and/or any other suitable graphical model having a temporal component. Any of such inference models may be trained using sensor signals obtained by the sensor(s) 110.

As another example, in some embodiments of the present technology, the inference model(s) 114 may be or may include a classifier that takes, as input, features derived from the sensor signals obtained by the sensor(s) 110. In such embodiments, the classifier may be trained using features extracted from the sensor signals. The classifier may be, e.g., a support vector machine, a Gaussian mixture model, a regression-based classifier, a decision-tree classifier, a Bayesian classifier, and/or any other suitable classifier, as aspects of the technology described herein are not limited in this respect. Input features to be provided to the classifier may be derived from the sensor signals in any suitable way. For example, the sensor signals may be analyzed as timeseries data using wavelet analysis techniques (e.g., continuous wavelet transform, discrete-time wavelet transform, etc.), covariance techniques, Fourier-analytic techniques (e.g., short-time Fourier transform, Fourier transform, etc.), and/or any other suitable type of time-frequency analysis technique. As one non-limiting example, the sensor signals may be transformed using a wavelet transform and the resulting wavelet coefficients may be provided as inputs to the classifier.

In some embodiments, values for parameters of the inference model(s) 114 may be estimated from training data. For example, when the inference model(s) is or includes a neural network, parameters of the neural network (e.g., weights) may be estimated from the training data. In some embodiments, parameters of the inference model(s) 114 may be estimated using gradient descent, stochastic gradient descent, and/or any other suitable iterative optimization technique. In embodiments where the inference model(s) 114 is or includes a recurrent neural network (e.g., an LSTM), the inference model(s) 114 may be trained using stochastic gradient descent and backpropagation through time. The training may employ any one or any combination of: a squared-error loss function, a correlation loss function, a cross-entropy loss function and/or any other suitable loss function, as aspects of the technology described herein are not limited in this respect.

The system 100 also may include one or more controller(s) 116. For example, the controller(s) 116 may include a display controller configured to display a visual representation (e.g., a representation of a hand) on a display device (e.g., a display monitor). As discussed herein, the processor(s) 112 may implement one or more trained inference model(s) that receive, as input, sensor signals obtained by the sensor(s) 110 and that provide, as output, information (e.g., predicted handstate information) used to generate control signals that may be used to control, for example, an AR or VR system.

The system 100 also may include a user interface 118. Feedback determined based on the signals obtained by the sensor(s) 110 and processed by the processor(s) 112 may be provided to the user via the user interface 119 to facilitate the user's understanding of how the system 100 is interpreting the user's muscular activity (e.g., an intended muscle movement). The user interface 118 may be implemented in any suitable way, including, but not limited to, an audio interface, a video interface, a tactile interface, and electrical stimulation interface, or any combination of the foregoing. The user interface 118 may be configured to produce a visual representation 108 (e.g., of a hand, an arm, and/or other body part(s) or a user), which may be displayed via a display device associated with the system 100.

As discussed herein, the computer processor(s) 112 may implement one or more trained inference model(s) configured to predict handstate information based, at least in part, on sensor signals obtained by the sensor(s) 110. The predicted handstate information may be used to update a musculoskeletal representation or model 106, which may be used to render the visual representation 108 (e.g., a graphical representation) based on the updated musculoskeletal representation or model 106. Real-time reconstruction of a current handstate and subsequent rendering of the visual representation 108 reflecting current handstate information in the musculoskeletal representation or model 106 may be used to provide visual feedback to the user about the effectiveness of the trained inference model(s), to enable the user to, e.g., make adjustments in order to represent an intended handstate accurately. As will be appreciated, not all embodiments of the system 100 include components configured to render the visual representation 108. For example, in some embodiments, handstate estimates output from the trained inference model and a corresponding updated musculoskeletal representation 106 may be used to determine a state of the user's hand (e.g., in a VR environment) even though no visual representation based on the updated musculoskeletal representation 106 is rendered.

The system 100 may have an architecture that may take any suitable form. Some embodiments of the present technology may employ a thin architecture in which the processor(s) 112 is or are included as a portion of a device separate from and in communication with the sensor(s) 110 arranged on one or more wearable device(s). The sensor(s) 110 may be configured to wirelessly stream, in substantially real-time, sensor signals and/or information derived from the sensor signals to the processor(s) 112 for processing. The device separate from and in communication with the sensors(s) 110 may be, for example, any one or any combination of: a remote server, a desktop computer, a laptop computer, a smartphone, a wearable electronic device such as a smartwatch, a health monitoring device, smart glasses, and an AR system.

Some embodiments of the present technology may employ a thick architecture in which the processor(s) 112 may be integrated with one or more wearable device(s) on which the sensor(s) 110 is or are arranged. In some embodiments, processing of sensed signals obtained by the sensor(s) 110 may be divided between multiple processors, at least one of which may be integrated with the sensor(s) 110, and at least one of which may be included as a portion of a device separate from and in communication with the sensor(s) 110. In such an implementation, the sensor(s) 110 may be configured to transmit at least some of the sensed signals to a first computer processor remotely located from the sensor(s) 110. The first computer processor may be programmed to train, based on the transmitted signals obtained by the sensor(s) 110, at least one inference model of the inference model(s) 114. The first computer processor may then be programmed to transmit the trained at least one inference model to a second computer processor integrated with the one or more wearable devices on which the sensor(s) 110 is or are arranged. The second computer processor may be programmed to determine information relating to an interaction between the user who is wearing the one or more wearable device(s) and a physical object in an AR environment using the trained at least one inference model transmitted from the first computer processor. In this way, the training process and a real-time process that utilizes the trained at least one model may be performed separately by using different processors.

In some embodiments of the present technology, a computer application configured to simulate an XR environment (e.g., a VR environment, an AR environment, and/or an MR environment) may be instructed to display a visual representation of the user's hand (e.g., via the controller(s) 116). Positioning, movement, and/or forces applied by portions of the hand within the XR environment may be displayed based on an output of the trained inference model(s). The visual representation may be dynamically updated based on current reconstructed handstate information using continuous signals obtained by the sensor(s) 110 and processed by the trained inference model(s) 114 to provide an updated computer-generated representation of the user's movement and/or handstate that is updated in real-time.

Information obtained by or provided to the system 100 (e.g., inputs from an AR camera, inputs from the sensor(s) 110 (e.g., neuromuscular sensor inputs), inputs from one or more auxiliary sensor(s) (e.g., IMU inputs), and/or any other suitable inputs) can be used to improve user experience, accuracy, feedback, inference models, calibration functions, and other aspects in the overall system. To this end, in an AR environment for example, the system 100 may include or may operate in conjunction with an AR system that includes one or more processors, a camera, and a display (e.g., the user interface 118, or another interface via AR glasses or another viewing device) that provides AR information within a view of the user. For example, the system 100 may include system elements that couple the AR system with a computer-based system that generates the musculoskeletal representation based on sensor data (e.g., signals from at least one neuromuscular sensor). In this example, the systems may be coupled via a special-purpose or other type of computer system that receives inputs from the AR system and the system that generates the computer-based musculoskeletal representation. Such a computer-based system may include a gaming system, robotic control system, personal computer, medical device, or another system that is capable of interpreting AR and musculoskeletal information. The AR system and the system that generates the computer-based musculoskeletal representation may also be programmed to communicate directly. Such information may be communicated using any number of interfaces, protocols, and/or media.

As discussed above, some embodiments of the present technology are directed to using an inference model 114 for predicting musculoskeletal information based on signals obtained by wearable sensors. Also as discussed briefly above, the types of joints between segments in a multi-segment articulated rigid body model constrain movement of the rigid body. The inference model 114 may be used to predict the musculoskeletal position information without having to place a sensor on each segment of the rigid body that is to be represented in the computer-generated musculoskeletal representation. Additionally, different individuals tend to move in characteristic ways when performing a task that can be captured in statistical or data patterns of individual user behavior. At least some of these constraints on human body movement may be explicitly incorporated in one or more inference model(s) (e.g., the model(s) 114) used for prediction of user movement, in accordance with some embodiments. Additionally or alternatively, the constraints may be learned by the inference model(s) 114 though training based on sensor data obtained from the sensor(s) 110. Constraints imposed on a construction of an inference model may be those set by human anatomy and by physics of a human body, while constraints derived from statistical or data patterns may be those set by human behavior for one or more users from which sensor data has been obtained. Constraints may comprise part of the inference model itself being represented by information (e.g., connection weights between nodes) in the inference model.

As mentioned above, some embodiments of the present technology are directed to using an inference model for predicting information to generate a computer-based musculoskeletal representation and/or to update in real-time a computer-based musculoskeletal representation. For example, the predicted information may be predicted handstate information. The inference model may be used to predict the handstate information based on IMU signals, neuromuscular signals (e.g., EMG, MMG, and/or SMG signals), external or auxiliary device signals (e.g., camera or laser-scanning signals), or a combination of IMU signals, neuromuscular signals, and external device signals detected as a user performs one or more movements. For instance, as discussed above, a camera associated with an AR system may be used to capture data of an actual position of a human subject of the computer-based musculoskeletal representation, and such actual-position information may be used to improve the accuracy of the representation. Further, outputs of the inference model may be used to generate a visual representation of the computer-based musculoskeletal representation in an XR environment. For example, a visual representation of muscle groups firing, force being applied, text being entered via movement, or other information produced by the computer-based musculoskeletal representation may be rendered in a visual display of an XR system. In some embodiments, other input/output devices (e.g., auditory inputs/outputs, haptic devices, etc.) may be used to further improve the accuracy of the overall system and/or to improve user experience. As mentioned above, XR may encompass any one or any combination of AR, VR, MR, and other machine-produced-reality technologies.

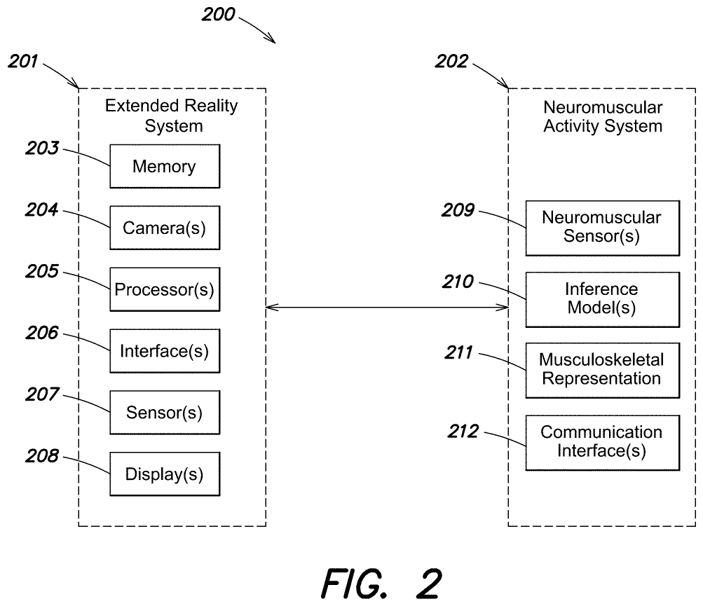

FIG. 2 illustrates a schematic diagram of an XR-based system 200 according to some embodiments of the present technology. The XR-based system may be a distributed computer-based system that integrates an XR system 201 with a neuromuscular activity system 202. The neuromuscular activity system 202 may be similar to the system 100 described above with respect to FIG. 1. As will be appreciated, instead of the XR system 201, the XR-based system 200 may comprise an AR system, a VR system, or a MR system.

Generally, the XR system 201 may take the form of a pair of goggles or glasses or eyewear, or other type of device that shows display elements to a user that may be superimposed on “reality.” This reality in some cases could be the user's view of the environment of his or her own body part(s) (e.g., arms and hands, legs and feet, etc., as viewed through the user's eyes), or those of another person or an avatar, or a captured view (e.g., by camera(s)) of the user's environment. In some embodiments, the XR system 201 may include one or more camera(s) 204, which may be mounted within a device worn by the user, that captures one or more views experienced by the user in the user's environment, including the user's own body part(s). The XR system 201 may have one or more processor(s) 205 operating within the device worn by the user and/or within a peripheral device or computer system, and such processor(s) 205 may be capable of transmitting and receiving video information and other types of data (e.g. sensor data). As discussed herein, captured video(s) of the user's body part(s) (e.g., hands and fingers) may be used as additional inputs to inference models, so that the inference models can more accurately predict the user's handstates, movements, and/or gestures. For example, information obtained from the captured video(s) can be used to train the inference models to recognize neuromuscular activation patterns or other motor-control signals, including by mapping or otherwise associating recorded images in the video(s) with the neuromuscular patterns detected during any one or more movement(s), gestures(,) and/or pose(s) as recorded.

The XR system 201 may also include one or more sensor(s) 207, such as microphones, GPS elements, accelerometers, infrared detectors haptic feedback elements, or any other type of sensor, or any combination thereof, that would be useful to provide any form of feedback to the user based on the user's movements and/or motor activities. In some embodiments, the XR system 201 may be an audio-based or auditory XR system, and the one or more sensor(s) 207 may also include one or more headphones or speakers. Further, the XR system 201 may also have one or more display(s) 208 that permit the XR system 201 to overlay and/or display information to the user in addition to the users' reality view. The XR system 201 may also include one or more communication interface(s) 206, which enable information to be communicated to one or more computer systems (e.g., a gaming system or other systems capable of rendering or receiving XR data). XR systems can take many forms and are provided by a number of different manufacturers. For example, various embodiments may be implemented in association with one or more types of XR systems or platforms, such as HoloLens™ holographic reality glasses available from the Microsoft Corporation (Redmond, Wash., USA); Lightwear™ AR headsets from Magic Leap™ (Plantation, Fla., USA); Google Glass™ AR glasses available from Alphabet (Mountain View, Calif., USA); R-7 Smartglasses System available from Osterhout Design Group (also known as ODG; San Francisco, Calif., USA); Oculus™ headsets (e.g., Quest, Rift, and Go) and/or Spark AR Studio gear available from Facebook (Menlo Park, Calif., USA); or any other type of XR device. Although discussed by way of example, it should be appreciated that one or more embodiments may be implemented within one type or a combination different types of XR systems (e.g., AR, MR, and/or VR systems).

The XR system 201 may be operatively coupled to the neuromuscular activity system 202 through one or more communication schemes or methodologies, including but not limited to: the Bluetooth protocol, Wi-Fi, Ethernet-like protocols, or any number of connection types, wireless and/or wired. It should be appreciated that, for example, the systems 201 and 202 may be directly connected or coupled through one or more intermediate computer systems or network elements. The double-headed arrow in FIG. 2 represents the communicative coupling between the systems 201 and 202.

As mentioned above, the neuromuscular activity system 202 may be similar in structure and function to the system 100 described above with reference to FIG. 1. In particular, the system 202 may include one or more neuromuscular sensor(s) 209, one or more inference model(s) 210, and may create, maintain, and store a musculoskeletal representation 211. In some embodiments of the present technology, similar to one discussed above, the system 202 may include or may be implemented as a wearable device, such as a band that can be worn by a user, in order to collect (i.e., obtain) and analyze neuromuscular signals from the user. Further, the system 202 may include one or more communication interface(s) 212 that permit the system 202 to communicate with the XR system 201, such as by Bluetooth, Wi-Fi, and/or another communication method. Notably, the XR system 201 and the neuromuscular activity system 202 may communicate information that can be used to enhance user experience and/or allow the XR system 201 to function more accurately and effectively. In some embodiments, the systems 201 and 202 may cooperate to determine a user's neuromuscular activity and to provide real-time feedback to the user regarding the user's neuromuscular activity.

Although FIG. 2 describes a distributed computer-based system 200 that integrates the XR system 201 with the neuromuscular activity system 202, it will be understood integration of these systems 201 and 202 may be non-distributed in nature. In some embodiments of the present technology, the neuromuscular activity system 202 may be integrated into the XR system 201 such that the various components of the neuromuscular activity system 202 may be considered as part of the XR system 201. For example, inputs of neuromuscular signals obtained by the neuromuscular sensor(s) 209 may be treated as another of the inputs (e.g., from the camera(s) 204, from the sensor(s) 207) to the XR system 201. In addition, processing of the inputs (e.g., sensor signals) obtained from the neuromuscular sensor(s) 209 as well as from one or more inference model(s) 210 can be performed by the XR system 201.

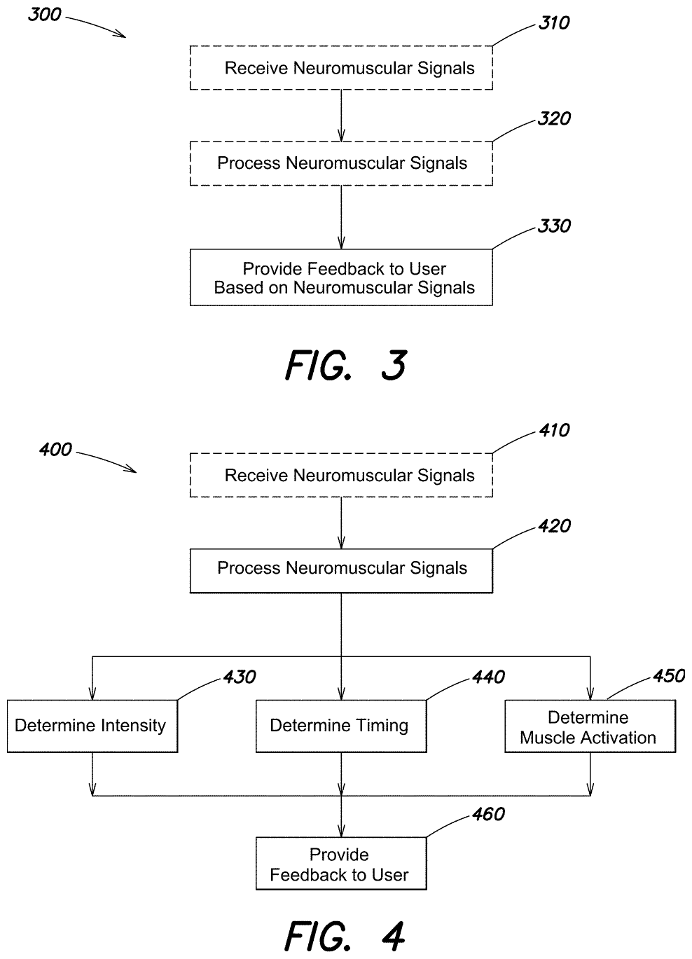

FIG. 3 shows a flowchart of a process 300 for using neuromuscular signals to provide feedback to a user, in accordance with some embodiments of the present technology. As discussed above, there are challenges involved with observation, detection, measurement, processing, and/or communication of neuromuscular activity. The systems and methods disclosed herein are capable of obtaining (e.g., detecting, measuring, and/or recording) and processing neuromuscular signals to determine muscular or sub-muscular activations (e.g., signal characteristics and/or patterns) and/or other suitable data from motor-unit and muscular activities, and providing feedback regarding such activations to the user. In some embodiments, a computer system may be provided along with one or more sensor(s) for obtaining (e.g., detecting, measuring, and/or recording) neuromuscular signals. As discussed herein, the sensor(s) may be provided on a band that can be placed on an appendage of the user, such as an arm or wrist of the user. In some embodiments, the process 300 may be performed at least in part by the neuromuscular activity system 202 and/or the XR system 201 of the XR-based system 200.

At block 310, the system obtains neuromuscular signals. The neuromuscular signals may comprise one or more muscular activation state(s) of the user, and these states may be identified based on raw signals obtained by one or more sensor(s) of the neuromuscular activity system 202 and/or processed signals (collectively “sensor signals”) and/or information based on or derived from the sensor signals (e.g., handstate information). In some embodiments, one or more computer processor(s) (e.g., the processor(s) 112 of the system 100, or the processor(s) 205 of the XR-based system 201) may be programmed to identify the muscular activation state(s) based on any one or any combination of: the sensor signals, the handstate information, static gesture information (e.g., pose information, orientation information), dynamic gesture information (movement information), information on motor-unit activity (e.g., information on sub-muscular activation), etc.

In some embodiments, the sensor(s) 209 of the neuromuscular activity system 202 may include a plurality of neuromuscular sensors 209 arranged on a wearable device worn by a user. For example, the sensors 209 may be EMG sensors arranged on an adjustable band configured to be worn around a wrist or a forearm of the user to sense and record neuromuscular signals from the user as the user performs muscular activations (e.g., movements, gestures). In some embodiments, the EMG sensors may be the sensors 1304 arranged on the band 1302, as shown in FIG. 13; in some embodiments, the EMG sensors may be the sensors 1410 arranged on the elastic band 1420, as shown in FIG. 14A. The muscular and/or sub-muscular activations performed by the user may include static gestures, such as placing the user's hand palm down on a table; dynamic gestures, such as waving a finger back and forth; and covert gestures that are imperceptible to another person, such as slightly tensing a joint by co-contracting opposing muscles, or using sub-muscular activations. The muscular activations performed by the user may include symbolic gestures (e.g., gestures mapped to other gestures, interactions, or commands, for example, based on a gesture vocabulary that specifies the mapping).

In addition to the plurality of neuromuscular sensors 209, in some embodiments of the technology described herein, the neuromuscular activity system 202 may include one or more auxiliary sensor(s) configured to obtain (e.g., sense and/or record) auxiliary signals that may also be provided as input to the one or more trained inference model(s), as discussed above. Examples of auxiliary sensors include IMUs, imaging devices, radiation detection devices (e.g., laser scanning devices), heart rate monitors, or any other type of biosensors able to sense biophysical information from a user during performance of one or more muscular activations. Further, it should be appreciated that some embodiments of the present technology may be implemented using camera-based systems that perform skeletal tracking, such as, for example, the Kinect™ system available from the Microsoft Corporation (Redmond, Wash., USA) and the LeapMotion™ system available from Leap Motion, Inc. (San Francisco, Calif., USA). It should be appreciated that any combination of hardware and/or software may be used to implement various embodiments described herein.

The process 300 then proceeds to block 320, the neuromuscular signals are processed. At block 330, feedback is provided to the user based on the processed neuromuscular signals. It should be appreciated that, in some embodiments of the present technology, the neuromuscular signals may be recorded; however, even in such embodiments, the processing and the providing of feedback may occur continuously, such that the feeding may be presented to the user in near real-time. Feedback that is provided in real-time or near real-time may be used advantageously in situations where the user is being trained, e.g., real-time visualizations provided to the user and/or a coach or trainer to train the user to perform particular movements or gestures properly. In some other embodiments, the neuromuscular signals may be recorded and analyzed at later times, and then presented to the user (e.g., during a review of a performance of a previous task or activity). In these other embodiments, the feedback (e.g., visualizations) may be provided much later, e.g. when analyzing a log of neuromuscular activity for the purposes of diagnoses and/or for tracking ergonomic/fitness/skill/compliance/relaxation. In skill-training scenarios (e.g. athletics, performing arts, industry), information regarding neuromuscular activity can be provided as feedback for training the user to perform one or more particular skill(s). In some cases, a target or desired pattern of neuromuscular activation may also be presented together with the feedback, and/or deviations of the user's actual or realized pattern from the target pattern may be presented or emphasized, such as by providing the user an auditory tone, a haptic buzz, a visual indication, template comparison feedback, or another indication. The target pattern for a task (e.g., a movement, etc.) may be produced from one or more previous pattern(s) of activation of the user or another person, such as during one or more instance(s) when the user or another individual performed particularly the task well (e.g., sat at a desk with his or her arms and hands in an ergonomic position to minimize wrist stain; threw a football or shot a basketball using proper technique; etc.). Further, it should be appreciated that comparison feedback to a target model or deviation information may be provided to the user in real-time, or later (e.g., in an offline review), or both. In certain embodiments, the deviation information can be used to predict an outcome of a task or activity, such as whether the user “sliced” a trajectory of a golf ball with a bad swing, hit a tennis ball with too much force and/or at too steep of an angle to cause the ball to land out-of-bounds, etc.

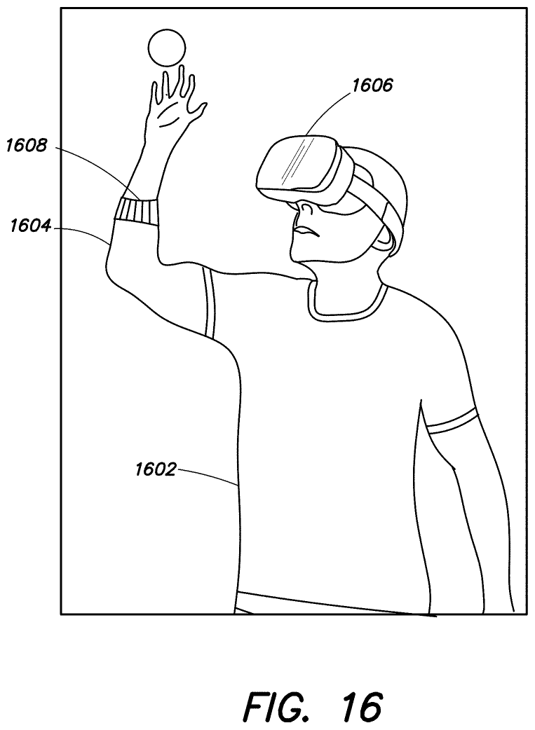

In some embodiments of the present technology, feedback is provided in the form of a visual display to convey musculoskeletal and/or neuromuscular activation information to a user. For instance, within an XR display, indications may be displayed to the user that identify a visualization of the activations or some other representation indicating that the neuromuscular activity performed by the user is acceptable (or not). In one example, in an XR implementation, visualization of muscular activation and/or motor-unit activation may be projected over the user's body. In this implementation, visualization of activated muscles within, e.g., an arm of the user may be displayed over the arm of the user within an XR display so the user can visualize various ranges of motions for his or her arm via an XR headset. For instance, as depicted in FIG. 16, a user 1602 may observe a visualization of muscular activations and/or motor-unit activations in the user's arm 1604 during throwing of a ball, by looking at the arm 1604 through an XR headset 1606 during the throwing. The activation are determined from the user's neuromuscular signals sensed by sensors of a wearable system 1608 (e.g., the wearable system 1400) during the throwing.

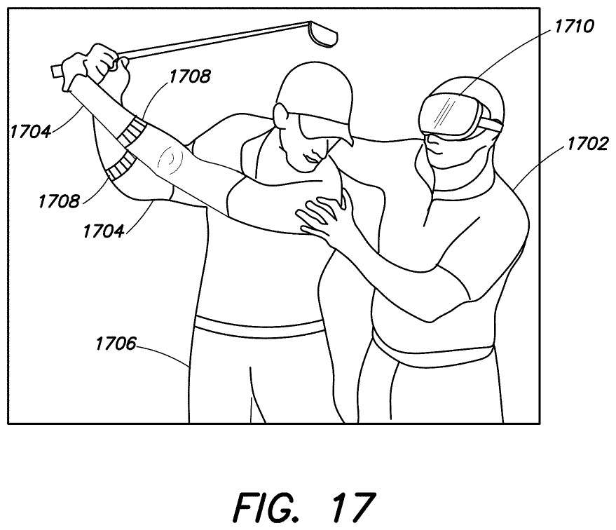

In another example, in an AR implementation, another person (e.g., a coach, a trainer, a physical therapist, an occupational therapist, etc.) may wear an AR headset to observe the user's activity while the user wears, e.g., an arm band on which neuromuscular sensors are attached (e.g., to observe while the user pitches a baseball, writes or draws on a canvas, etc.). For instance, as depicted in FIG. 17, a coach 1702 may observe a visualization of muscular activations and/or motor-unit activations in one or both arm(s) 1704 of a golfer 1706 during swinging of a golf club by the golfer. The activations are determined from the golfer's neuromuscular signals sensed by sensors of a wearable system 1708 (e.g., the wearable system 1400) worn by the golfer 1706. The visualizations may be seen by the coach 1702 via a AR headset 1710.

In some embodiments of the present technology, the feedback may be visual and may take many one or more form(s), and may be combined with other types of feedback, such as non-visual feedback. For instance, auditory, haptic, electrical, or other feedback may be provided to the user in addition to visual feedback.

FIG. 4 shows a flowchart of a process 400 in which neuromuscular signals are used to determine intensity, timing, and/or occurrence of one or more muscle activation(s), in accordance with some embodiments of the technology described herein. Systems and methods according to these embodiments may help overcome the difficulty in observing, describing, and/or communicating about neuromuscular activity, such as a timing and/or an intensity of motor-unit and/or muscle activations. Skilled motor acts may require precise coordinated activations of motor units and/or muscles, and learning to perform skilled acts may be hindered by difficulties with observing and communicating about such activations. Further, difficulty communicating about such activations can be a hindrance to coaches and medical providers. As will be appreciated, feedback regarding a person's performance of skilled motor acts is needed in neuromuscular control technology, where the person may use neuromuscular signals to control one or more devices.

In some embodiments of the present technology, the process 400 may be performed at least in part by a computer-based system such as the neuromuscular activity system 202 and/or the XR system 201 of the XR-based system 200. More specifically, neuromuscular signals may be obtained from a user wearing one or more neuromuscular sensor(s), and, at block 410, the neuromuscular signals may be received by the system. For example, the sensor(s) may be arranged on or within a band (e.g., the bands of the wearable systems 1300 and 1400) and positioned over an area of the user's body, such as an arm or a wrist. At block 420, the received neuromuscular signals are processed to determine one or more aspects of these signals. For example, at block 430, the system may determine an intensity of an activation (e.g., a contraction) of a particular motor unit or an intensity of one or more group(s) of motor units of the user. In this example, the system may determine a firing rate of the motor unit(s) and/or associated force(s) generated by the motor unit(s). The system may provide information about the determined intensity as feedback to the user, at act 460, and this feedback may be provided alone or in combination with other information derived from the neuromuscular signals. At block 440, the system may determine a timing of activities of a particular motor unit. In certain embodiments, maximal muscular activation or contraction states of a particular user can be previously recorded and used as a comparator to current muscular activation or contraction states of the user as detected and recorded during the user's performance of a movement or exercise. For example, if the user's maximal velocity for throwing a baseball is 100 mph, i.e., a fastball, the muscular activation or contraction states of the user's arm and shoulder muscles, as detected during such throwing of a fastball, can be used to visually compare the previously recorded muscular-activation or contraction states with the currently recorded states during the user's successive performances of throwing a fastball. In another example, a user with motor neuropathy can be monitored in real-time during treatment by a medical provider by comparing previously recorded forearm muscular activation states with current muscular activation states detected as the user, e.g., draws on a canvas, and such real-time comparison feedback of current versus previous muscular activation states can be presented to the user and/or the medical provider. At block 440, the system may also determine a timing of one or more particular motor-unit activation(s). For example, how the motor unit(s) function over a period of time may be determined from the neuromuscular signals, and feedback regarding such a timing determination may be provided to the user (e.g., at block 460). For instance, a sequence and timing of activities of particular motor unit(s) may be presented to the user, alone or in conjunction with model or target information previously collected from the user or from a different person. Also, specific information relating to, e.g., one or more particular muscle activation(s) may be determined at block 450 and presented to the user as feedback at block 460. As will be appreciated, the blocks 430, 440, and 450 may be performed concurrently or sequentially or, in some embodiments, only one or two of these acts may be performed while the other one or two of these acts may be omitted.

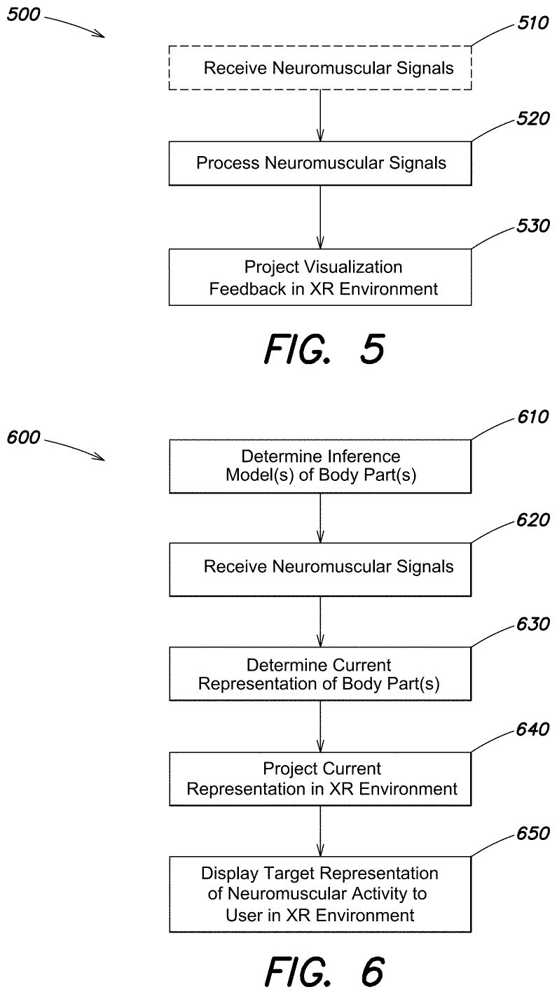

FIG. 5 shows a flowchart of a process 500 in which neuromuscular signals are processed to produce a visualization, which may be projected in an XR environment, in accordance with some embodiments of the technology presented herein. In particular, in the XR environment, the visualization may be projected over a body part of the user, such as an arm of the user, to provide the user with feedback information that may involve the body part. For instance, in one implementation, the projection may include a visual indication that shows muscle-group activations and/or degrees of joint angles within the projected feedback information. In one such scenario, a muscular representation (e.g., an animated view of muscle activations) may be projected over a view of the user's arm, and indications of particular activations and/or joint angles as measured by the received and processed neuromuscular signals may be shown by the muscular representation. The user then may adjust his/her movement to achieve a different result. In an exercise scenario, the user may use the XR visualization as feedback to slightly vary his/her intensity or movement to achieve a desired muscle activation (e.g., to activate a certain muscle group to be exercised) at a certain intensity level) and may do so at a given joint angle as provided in the feedback. In this way, the user can monitor and control the intensity of his or her muscular activation(s) or track ranges of motion of one or more joints. It can be appreciated that such feedback would be advantageous in other scenarios, including but not limited to: physical rehabilitation scenarios where the user works to strengthen muscles and/or surrounding ligaments, tendons, tissues, etc., or to increase a joint's range of motion; athletic performance scenarios, such as throwing a baseball, shooting a basketball, swinging a golf club or tennis racquet, etc.; and coaching or instructional scenarios where another person alone, or in combination with the user, views the user's muscular activation and/or joint-angle feedback and provides corrective instruction to the user.

FIG. 6 shows a flowchart for a process 600 in which neuromuscular signals are processed to produce a visualization, which may be displayed in an XR environment, in accordance with some embodiments of the present technology. In particular, the process 600 may be executed to enable the user to view a visualization of a target or desired neuromuscular activity within the XR environment as well as a visualization of a realized neuromuscular activity performed by the user. The process 600 may be executed at least in part by a computer-based system such as the neuromuscular activity system 202 and/or the XR system 201 of the XR-based system 200. In skill-training scenarios (e.g. athletics, performing arts, industry, etc.), information regarding a target neuromuscular activity can be provided as extra feedback for the user. In some cases, a target pattern of neuromuscular activation may be presented to the user in a display (e.g., within an XR display, or another type of display) and/or deviations of a realized pattern obtained from the user's neuromuscular signals from the target pattern may be presented or emphasized. Such deviations may be presented to the user in one or more form(s), such as an auditory tone, a haptic buzz, a visual indication (e.g., a visual representation of the realized pattern superimposed to a visual representation of the target pattern in which deviations are highlighted), and the like. It can be appreciated that in some instances deviations between the realized pattern and the targeted pattern can be generated and provided to the user in real-time or near real-time, while in other instances such deviations can be provided “offline” or after fact, such as upon the user's request at a later time.

One way to create the target pattern may be from one or more previously performed realized pattern(s) of activation during one or more instance(s) when the user or another individual performed a desired activation task particularly well. For example, in one scenario, an expert (e.g., an athlete) may perform the desired activation task well, and neuromuscular signals may be obtained from the expert during performance of that task. The neuromuscular signals may be processed to obtain visual target neuromuscular activations, which may be displayed as feedback to the user within, e.g., a display in an XR environment. In various embodiments of the present technology, the feedback can be shown to the user as a separate example display, as activations that are grafted or projected onto the user's appendage(s), and/or as activations that may be compared to the user's actual or realized activations.

In FIG. 6, at block 610, the system determines an inference model built according to the user's body or body part (e.g., hand, arm, wrist, leg, foot, etc.). The inference model may be or may include one or more neural network model(s), as discussed above, trained to classify and/or assess neuromuscular signals captured from a user. The inference model may be trained to recognize one or more pattern(s) that characterize a target neuromuscular activity. At block 620, the system receives neuromuscular signals from one or more sensor(s) worn by the user during performance of a task corresponding to the target neuromuscular activity, and at block 630, the system determines a current representation of one or more part(s) of the user's body (e.g., appendage(s) and/or other body part(s)) based on the received neuromuscular signals and the inference model.

At block 640, the system projects the current representation of the user's body part(s) within the XR environment. For example, the XR display may display a graphical representation of the user's body over an actual view of the body part(s) (e.g., of an arm) or an avatar can be presented that mimics the user's appearance in the XR environment. Further, neuromuscular status information may be displayed within this representation, such an indication of muscular activity within one or more muscle groups. At block 650, the XR display may also display a target representation of neuromuscular activity. For instance, the target representation may be displayed on the same display as the current representation of the user's body part(s), and may be shown as an image that is projected onto a view of the user, e.g., an actual appendage of the user or onto the user's avatar or through some other representation of the user's appendage, which need not connect directly to the user. As discussed above, such feedback may be provided to the user by itself or in combination with other types of feedback indicating the user's performance of the task, such as haptic feedback, audio feedback, and/or other types of feedback.

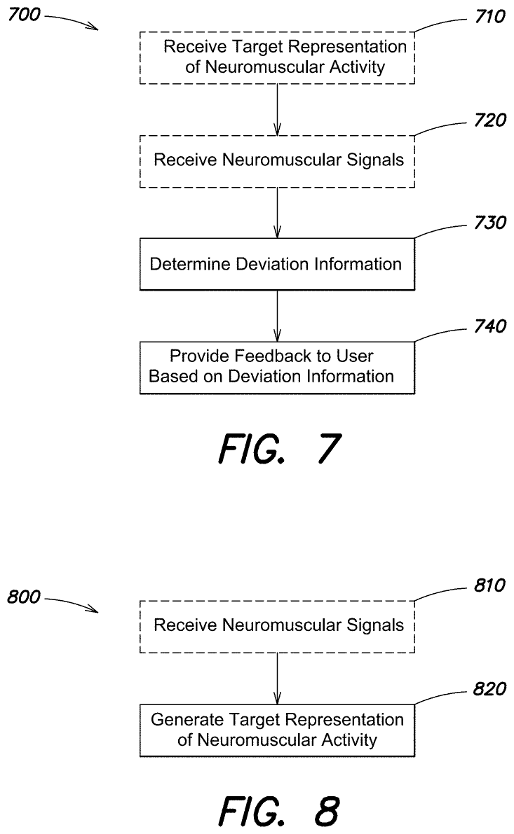

FIG. 7 shows a flowchart for another process 700 in which neuromuscular signals, which are obtained from a user during performance of a task (e.g., a movement), are processed to determine deviations of the user's performance from a target performance, and providing feedback to the user in the form of deviation information, in accordance with some embodiments of the present technology. Such deviation information, resulting from the process 700, may help the user achieve or perform, e.g., a desired movement that closely resembles the target performance. In one implementation, deviation information may be input into the system automatically and may be derived from previously processed inputs relating to a correct or best way of performing a given task, activity, or movement. In another implementation, in addition to or alternative to the automatically input deviation information, deviation information may be manually input by the user to help the user achieve movement(s) closer to a target for the given task, activity, or movement. For instance, deviations of a realized pattern, determined from the user's performance, from a target pattern, corresponding to a target performance, may be presented or emphasized to the user as feedback in the form of, e.g., an auditory tone that increases in loudness according to a deviation amount, a haptic buzz that increases in amplitude according to the deviation amount, or a visual indication showing the deviation amount) and/or the user can update deviation information manually by, e.g., make a drawing or an annotation within the XR environment.

The process 700 may be executed at least in part by a computer-based system such as the neuromuscular activity system 202 and/or the XR system 201 of the XR-based system 200. At block 710, the system may receive a target representation of neuromuscular activity. For instance, the target representation may identify a target movement and/or one or more target muscle activation(s). The target representation of neuromuscular activity may be a recorded signal provided to the system and used as a reference signal. At block 720, the system may receive neuromuscular signals obtained from a user wearing one or more neuromuscular sensor(s) while performing an act (e.g., a movement, a gesture, etc.) to be evaluated. For instance, the user may wear a band (e.g., the bands in FIGS. 13 and 14A) carrying sensors that sense the neuromuscular signals from the user and provides the sensed neuromuscular signals to the system in real time and provide feedback to the user (in real time, near-real time, or at a later period (e.g., in a review session)). At block 730, the system may determine deviation information derived by comparing a target activity to a measured activity based on the received neuromuscular signals. The feedback provided to the user may include parameters that determine a quality measure of an entire act performed by the user (e.g., a complex movement comprising multiple muscle activations and/or physical movement) and/or specific elements of the act (e.g., a specific muscle activation). In some embodiments, joint angles, motor-unit timing(s), intensity(ies), and/or muscle activation(s) relating to the user's neuromuscular activations may be measured in relation to the target activity. In particular, comparisons may be performed between models (e.g., a target model and a user model to be evaluated). Further, in some embodiments, the target model may be adapted to specifics of the user model to provide more accurate comparisons (e.g., normalizing the target model to a specific user based on differences in sizes between the user and a model performer of the target model).

At block 740, feedback can be provided to the user based on the deviation information. In particular, the deviation information may indicate to the user that an activity or task was performed correctly or incorrectly, or was performed to some measured quality within a range. Such feedback may be visual, such as by an indication within an XR display that a particular muscle group was not activated via a projection on the user's arm (e.g., a projection of a muscle group colored red on the user's arm) or that the particular muscle group was only partially activated (e.g., activated to 75% as opposed to an intended 90% of maximal contraction). Also, a display of timing(s), intensity(ies), and/or muscle activation(s) relating to the user's neuromuscular activations may be displayed to the user within the XR display (e.g., as projection onto the user's body or onto the user's avatar). As discussed above, the visual feedback may be provided alone or in combination with other feedback, such as auditory (e.g., by a voice indication that the user's movement is unsatisfactory), haptic (such as a haptic buzz, resistive tension, etc.), and/or other feedback. Such deviation information may be helpful for the user to improve his or her performance of the activity or task and to more accurately track the target activity. This type of feedback could assist users developing their ability to use control systems involving neuromuscular signals. For example, visualization of neuromuscular activations could help a user learn to activate atypical combinations of muscles or motor units.

FIG. 8 shows a flowchart for a process 800 for generating a target neuromuscular activity based on received neuromuscular signals, in accordance with some embodiments of the present technology. The process 800 may be executed at least in part by a computer-based system such as the XR-based system 200. As discussed above, the system may use a target activity as a reference by which the user's activity may be assessed or measured. To elicit such a target activity, a neuromuscular system or other type of system (e.g., the neuromuscular activity system 202) may receive neuromuscular signals (e.g., at block 810) and may generate a model of a target neuromuscular activity based on these signals. Such neuromuscular signals may be used in addition to other types of signals and/or data such as, for example, camera data. Such neuromuscular signals may be sampled from an expert performer (e.g., an athlete, a trainer, or another suitably skilled person) and modeled for use as the target activity. For instance, a golf swing activity may be captured from one or more golfing professional(s), modeled, and stored as a target activity for use in a golf training exercise, game, or other system.

In some instances, neuromuscular signals sampled from the user's previous performances of an activity can be used to assess user's progress over time, based on computed deviations between user's previous performances and a current performance of the user (e.g., for training and/or rehabilitation over time). In this way, the system can track the user's performance progress in relation to a reference activity.

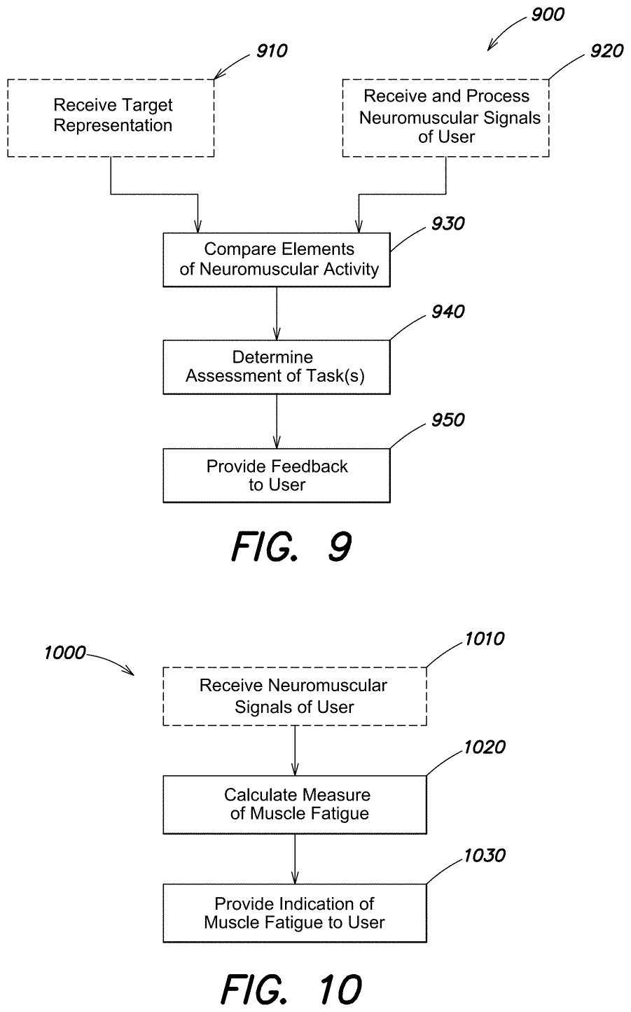

FIG. 9 shows a flowchart for a process 900 for assessing one more task(s) based on compared neuromuscular activity, in accordance with some embodiments of the present technology. The process 900 may be executed at least in part by a computer-based system such as the XR-based system 200. As discussed above, inference models may be trained and used to model a user's neuromuscular activity as well as a target or model activity. Also, as discussed above with reference to FIG. 8, the system may be capable of receiving a target neuromuscular activity (e.g., at block 910) to be used as a reference. Such target activity may be preprocessed and stored in memory (e.g., within a processing system, a wearable device, etc.) for future comparisons. At block 920, the system may receive and process neuromuscular signals of the user being monitored. For example, sensors of a wearable system (e.g., 1300 shown in FIG. 13, 1400 shown in FIG. 14) may be worn by the user to sense the neuromuscular signals from the user, and the neuromuscular signals may be provided to the system for processing (e.g., processing via one or more inference model(s), as discussed above). At block 930, the system may compare elements of neuromuscular activity from the sensed signals to the stored reference.

At block 940, the system may determine an assessment of one or more task(s). The assessment may be an overall assessment of a complex movement and/or an assessment of one or more specific element(s), such as a muscle movement. At block 950, feedback may be provided to the user by the system (e.g., in an XR display with or without other feedback channels, as described above).

In some implementations, the feedback provided to the user be provided in real-time or near real-time, as is advantageous for training. In other implementations, the feedback (e.g., a visualization) may be provided at a later time, e.g., when analyzing a log of neuromuscular activity for purposes of diagnoses and/or for ergonomic/fitness/skill/compliance/relaxation tracking. In some embodiments, such as monitoring (in real-time) a compliance-tracking task, the user may receive feedback in near real-time. For example, the user may be instructed to tighten a screw, and, based on the user's neuromuscular activity, the system could estimate how tightly the user turned the screw and provide feedback to adjust his or her performance of this task accordingly (e.g., by presenting text and/or an image in an XR environment signaling that the user needs to continue tightening the screw). Further, although a target activity may require a high level of skill to be performed well (e.g., to hit a golf ball accurately), it should be appreciated that the system may be used to measure any activity requiring any level of skill.

In some embodiments of the technology described herein, information about the user's muscle activations may be available long before the user would otherwise get feedback about his or her performance of a task corresponding to the muscle activations. For example, a golfer may have to wait multiple seconds for an outcome of a swing (e.g., waiting to see whether a ball hit by the golfer deviates from a desired trajectory), and a tennis player may have to wait for an outcome of a swing (e.g., waiting to see ball to hit the ground before learning whether a serve was in play or out of hounds). In cases such as these, the system may present immediate feedback derived from neuromuscular data (possibly in conjunction with other data such as that from one or more auxiliary sensor(s)), for example, a tone to indicate that the system has detected that the serve will land out of bounds. Advance feedback such as this can be used to, e.g., abort a performance of the task when permissible (e.g., if an error is detected during the golfer's backswing) or to facilitate training with more immediate feedback. The system can be trained, for example, by having the user indicate (e.g., with voice) whether each instance of a motor act (a completed golf swing in this example) was successful, to provide supervised training data.

In some embodiments, feedback presented to a user during performance of a task or after completion of the task may relate to the user's ability to perform the task accurately and/or efficiently. For example, neuromuscular signals recorded during a performance of the task (e.g., tightening a bolt) may be used to determine whether the user performed the task accurately and/or optimally, and feedback may be provided to instruct the user about how to improve performance of the task (e.g., provide more force, position hands and/or fingers in an alternate configuration, adjust hands and/or arms and/or fingers relative to each other, etc.). In some embodiments, the feedback regarding the performance of the task may be provided to the user before the task has been completed, in order to guide the user through proper performance of the task. In other embodiments, the feedback may be provided to the user, at least in part, after the task has been completed to allow the user to review his or her performance of the task, in order to learn how to perform the task correctly.

In some other embodiments relating to physical skill training, augmentation and instrumentations, the system may be used to monitor, assist, log, and/or help the user in a variety of scenarios. For example, the system may be used in a following (e.g., counting) activity, such as knitting or an assembly-line activity. In such cases, the system may be adapted to follow along the user's movements, align his or her activities with instruction(s), step(s), pattern(s), recipe(s), etc.

Further, the system may be adapted to provide error detection and/or alerting functions. For instance, the system can prompt the user with help, documents, and/or other feedback to make the user more efficient and to keep the user on track with performing a task. After the task has been performed, the system may compute metrics about task performance (e.g., speed, accuracy).

In some embodiments of the present technology, the system may be capable of providing checklist monitoring to assist the user in performing an overall activity or set of tasks. For instance, surgeons, nurses, pilots, artists, etc., who perform some types of activities may benefit by having an automated assistant that is capable of determining whether certain tasks for an activity were performed correctly. Such a system may be capable of determining whether all tasks (e.g., physical-therapy steps) on a checklist were executed properly, and may be capable of providing some type of feedback to the user that tasks on the checklist were completed.

Aspects described herein may be used in conjunction with control assistants. For instance, control assistants may be provided for smoothing input actions of the user in order to achieve a desired output control, such as within a surgical mechanical device to smooth shaky hands (e.g., Raven), within a CAD program (e.g., AutoCAD) to control drafting input, within a gaming application, as well as within some other type(s) of applications.

Aspects described herein may be used in other applications such as life logging applications or other applications where activity detection is performed and tracked. For instance, various elements may be implemented by systems (e.g., activity trackers such as Fitbit® available from Fitbit, Inc., (San Francisco, Calif., USA), and the like) that can detect and recognize different activities, such as eating, walking, running, biking, writing, typing, brushing teeth, etc. Further, various implementations of such system may be adapted to determine, e.g., how often, how long, how much the recognized activities were performed. The accuracy of such systems may be improved using neuromuscular signals, as neuromuscular signals may be more accurately interpreted than existing inputs recognized by these systems. Some further implementations of such systems may include applications that assist users to learn physical skills. For example, a user's performance of activities requiring physical skills such as performing music, athletics, controlling a yoyo, knitting, magic tricks, etc.) may be improved by a system that can detect and provide feedback on the user's performance of such skills. For instance, in some implementations, the system may provide visual feedback and/or feedback that may be presented to the user in a gamified form. In some implementations, feedback may be provided to the user in the form of coaching (e.g., by an artificial-intelligence inference engine and/or an expert system), which may assist the user in learning and/or performing a physical skill.

FIG. 10 shows a flowchart for process 1000 for monitoring muscle fatigue, in accordance with some embodiments of the technology described herein. In particular, it is realized that there may be a benefit in observing muscle fatigue in a user and providing indications of muscle fatigue to the user (or to another system, or to another person (e.g., trainer), etc.). The process 1000 may be executed at least in part by a computer-based system such as the XR-based system 200. At block 1010, the system receives neuromuscular signals of the user being monitored via one or more sensor(s) (e.g., on the wearable systems 1300 and 1400 shown in FIGS. 13 and 14A, or another sensor arrangement). At block 1020, the system may calculate or determine a measure of muscle fatigue from the user's neuromuscular signals. For instance, fatigue may be calculated or determined as a function of spectral changes in EMG signals over time, using historical neuromuscular signals collected for the user. Alternatively, fatigue may be assessed based on a firing pattern of one or more motor unit(s) of the user. Other methods for calculating or determining fatigue based on neuromuscular signals may be used, such as an inference model that translates neuromuscular signals into a subjective fatigue score. At block 1030, the system may provide an indication of muscle fatigue to the user (or to another system, a third party (e.g., a trainer, medical provider), or another entity (e.g., a vehicle that monitors muscle fatigue)). For instance, the indication can be provided visually (e.g., by a projection in an XR environment, or another type of visual indication), audibly (e.g., a voice indicating fatigue is occurring), or another type of indication. In this way, more detailed information regarding the user may be collected and presented as feedback.

For example, in safety or ergonometric applications, the user may be provided with immediate feedback (e.g., a warning) indicating, e.g., muscle activation and fatigue level, which can be detected by spectral changes in data captured by EMG sensors or another suitable type of sensor, and the user also may be provided with an historical view of a log of the user's muscle activations and fatigue levels, potentially within a postural context. The system may provide as feedback a suggestion to change a technique (for physical tasks) or to change a control scheme (for virtual tasks) as a function of the user's fatigue. For instance, the system may be used to alter a physical rehabilitation training program, such as by increasing an amount of time to a next session, based on a fatigue score determined within a current rehabilitation session. A measure of fatigue may be used in association with other indicators to warn the user or others of one or more issue(s) relating to the user's safety. For instance, the system may help in determining ergonometric issues (e.g., to detect whether the user is lifting too much weight, or typing inappropriately or with too much force, etc.) and recovery monitoring (e.g., to detect whether the user is pushing himself or herself too hard after an injury). It should be appreciated that various embodiments of the system may use fatigue level as an indicator or as input for any purpose.

In some embodiments of the present technology, systems and methods are provided for assisting, treating, or otherwise enabling a patient with an injury or a disorder that affects his or her neuromuscular system by delivering feedback about the patient's neuromuscular activity (i.e. in an immersive experience such as via XR displays, haptic feedback, auditory signals, user interfaces, and/or other feedback types to assist the patient in performing certain movements or activities). For a patients taking part in neuro-rehabilitation, which may be required due to injury (e.g., peripheral nerve injury and/or spinal cord injury), stroke, cerebral palsy, or another cause, feedback about patterns of neuromuscular activity may be provided that permit the patients to gradually increase neuromuscular activity or otherwise improve their motor-unit outputs. For example, a patient may only be able to activate a small number of motor units during an early phase of therapy, and the system may provide feedback (e.g., ‘high-gain feedback’) showing a virtual or augmented part of the patient's body moving a greater degree than actually occurs. As therapy progresses, the gain provided for feedback can be reduced as the patient achieves better motor control. In other therapeutic examples, a patient may have a motor disorder such as a tremor and be guided through feedback specific to the patient's neuromuscular impairment (e.g. that shows less tremor in the feedback). Thus, feedback may be used to show small incremental changes in neuromuscular activation (e.g., each increment being recognized as achievable by the patient), to encourage the patient's rehabilitation progress.

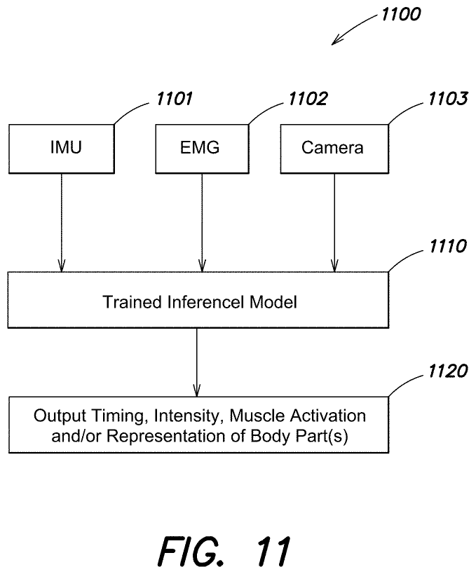

FIG. 11 shows a flowchart of a process 1100 in which inputs are provided to a trained inference model, in accordance with some embodiments of the technology described herein. For example, the process 1100 may be executed at least in part by a computer-based system such as the XR-based system 200. In various embodiments of the present technology, a more accurate musculoskeletal representation may be obtained by using IMU inputs (1101), EMG inputs (1102), and camera inputs (1103). Each of these inputs may be provided to a trained inference model 1110. The inference model may be capable of providing one or more outputs such as position, force, and/or a representation of the musculoskeletal state. Such outputs may be utilized by the system or provided to other systems to produce feedback for the user. It should be appreciated that any of the inputs may be used in any combination with any other input to derive any output, either alone or in combination with any output list or any other possible output. For instance, forearm positional information may be derived based on a combination of IMU data and camera data. In one implementation, an estimate of forearm position may be generated based on IMU data and adjusted based on ground-truth camera data. Also, forearm position and/or forearm orientation may be derived using camera data alone without IMU data. In another scenario, EMG signals may be used to derive force-only information to augment posture-only information provided by a camera-model system. Other combinations of inputs and output are possible and within the scope of various embodiments descried herein.

It should also be appreciated that such outputs may be derived with or without generating any musculoskeletal representation. It should also be appreciated that one or more outputs may be used as control inputs to any other system, such as an EMG-based control that is used to control an input mode of an XR system, or vice-versa.

It is appreciated that any embodiment described herein may be use alone or in any combination with any other embodiment described herein. Further embodiments are described in more detail in U.S. patent application Ser. No. 16/257,979 filed Jan. 25, 2019, entitled “CALIBRATION TECHNIQUES FOR HANDSTATE REPRESENTATION MODELING USING NEUROMUSCULAR SIGNALS,” which is incorporated by reference herein in its entirety.

The above-described embodiments can be implemented in any of numerous ways. For example, the embodiments may be implemented using hardware, software or a combination thereof. When implemented using software, code comprising the software can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments of the technologies described herein. The computer-readable storage medium can be transportable such that the program stored thereon can be loaded onto any computer resource to implement the aspects of the present invention discussed herein. In addition, it should be appreciated that reference to a computer program that, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the technology presented herein may be used alone, in combination, or in a variety of arrangements not specifically discussed in the embodiments described above and therefore are not limited in their application to the details and arrangements of components set forth in the foregoing description and/or in the drawings.

Also, some of the embodiments described above may be implemented as one or more method(s), of which some examples have been provided. The acts performed as part of the method(s) may be ordered in any suitable way. Accordingly, embodiments may be constructed in which acts are performed in an order different than illustrated or described herein, which may include performing some acts simultaneously, even though shown as sequential acts in illustrative embodiments. The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

The foregoing features may be used, separately or together in any combination, in any of the embodiments discussed herein.

Further, although advantages of the present invention may be indicated, it should be appreciated that not every embodiment of the invention will include every described advantage. Some embodiments may not implement any features described as advantageous herein. Accordingly, the foregoing description and attached drawings are by way of example only.

Variations on the disclosed embodiment are possible. For example, various aspects of the present technology may be used alone, in combination, or in a variety of arrangements not specifically discussed in the embodiments described in the foregoing, and therefore they are not limited in application to the details and arrangements of components set forth in the foregoing description or illustrated in the drawings. Aspects described in one embodiment may be combined in any manner with aspects described in other embodiments.

Use of ordinal terms such as “first,” “second,” “third,” etc., in the description and/or the claims to modify an element does not by itself connote any priority, precedence, or order of one element over another, or the temporal order in which acts of a method are performed, but are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use of the phrase “equal” or “the same” in reference to two values (e.g., distances, widths, etc.) means that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Also, the phraseology and terminology used herein is for the purpose of description and should not be regarded as limiting. Use of terms such as “including,” “comprising,” “comprised of,” “having,” “containing,” and “involving,” and variations thereof herein, is meant to encompass the items listed thereafter and equivalents thereof as well as additional items.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A computerized system for providing feedback to a user based on neuromuscular signals sensed from the user, the system comprising:
a plurality of neuromuscular sensors configured to sense a plurality of neuromuscular signals from the user, wherein the plurality of neuromuscular sensors are arranged on one or more wearable devices; and
at least one computer processor programmed to:
process the plurality of neuromuscular signals using one or more inference or statistical models, and
provide feedback to the user based on one or both of:
the processed plurality of neuromuscular signals, and
information derived from the processed plurality of neuromuscular signals,
wherein the feedback comprises visual feedback that includes information relating to one or both of:
a timing of an activation of at least one motor unit of the user, and an intensity of the activation of the at least one motor unit of the user.

2. The computerized system of claim 1, wherein
the feedback provided to the user comprises auditory feedback, or haptic feedback, or both auditory feedback and haptic feedback, and
the auditory feedback and the haptic feedback relate to one or both of:
the timing of the activation of the at least one motor unit of the user, and
the intensity of the activation of the at least one motor unit of the user.

3. The computerized system of claim 1, wherein
the visual feedback further includes a visualization relating to one or both of: the timing of the activation of the at least one motor unit of the user, and the intensity of the activation of the at least one motor unit of the user,
the visualization is provided within an augmented reality (AR) environment generated by an AR system or a virtual reality (VR) environment generated by a VR system, and
the visualization depicts at least one body part, the at least one body part comprising any one or any combination of:
a forearm of the user, a
wrist of the user, and a
leg of the user.

4. The computerized system of claim 1, wherein
the at least one computer processor is programmed to predict an outcome of a task or activity performed by the user based, at least in part, on one or both of: the plurality of neuromuscular signals and information derived from the plurality of neuromuscular signals, and
the feedback provided to the user comprises an indication of the predicted outcome.

5. The computerized system of claim 4, wherein the task or activity is associated with an athletic movement or a therapeutic movement.

6. The computerized system of claim 3, wherein the at least one computer processor is programmed to provide to the user a visualization of at least one target neuromuscular activity state, the at least one target neuromuscular activity state being associated with performing a particular task.

7. The computerized system of claim 6 wherein
the at least one computer processor is programmed to determine, based on one or both of: the plurality of neuromuscular signals and information derived from the plurality of neuromuscular signals, deviation information from the at least one target neuromuscular activity state, and
the feedback provided to the user comprises feedback based on the deviation information.

8. The computerized system of claim 3, wherein
the at least one computer processor is further programmed to calculate a measure of muscle fatigue from one or both of: the plurality of neuromuscular signals and information derived from the plurality of neuromuscular signals, and
the visual feedback provided to the user comprises a visual indication of the measure of muscle fatigue.

9. A computerized system for providing feedback to a user based on neuromuscular signals sensed from the user, the system comprising:
a plurality of neuromuscular sensors configured to sense a plurality of neuromuscular signals from the user, wherein the plurality of neuromuscular sensors are arranged on one or more wearable devices: and
at least one computer processor programmed to:
process the plurality of neuromuscular signals using one or more inference or statistical models, and
provide feedback to the user based on the processed plurality of neuromuscular signals,
wherein the feedback provided to the user is associated with one or more neuromuscular activity states of the user, and
wherein the plurality of neuromuscular signals relate to an athletic movement or a therapeutic movement performed by the user.

10. The computerized system of claim 9, wherein the feedback provided to the user comprises any one or any combination of: audio feedback, visual feedback, and haptic feedback.

11. The computerized system of claim 9, wherein the feedback provided to the user comprises visual feedback within an augmented reality (AR) environment generated by an AR system or a virtual reality (VR) environment generated by a VR system.

12. The computerized system of claim 11, wherein
the visual feedback provided to the user comprises a visualization of one or both of:
a timing of an activation of at least one motor unit of the user, and
an intensity of the activation of the at least one motor unit of the user, and the visualization depicts at least one body part, the at least one body part comprising
any one or any combination of:
a forearm of the user, a
wrist of the user, and a
leg of the user.

13. The computerized system of claim 11, wherein
the at least one computer processor is further programmed to provide to the user a visualization of at least one target neuromuscular activity state, and
the at least one target neuromuscular activity state is associated with performing the athletic movement or the therapeutic movement.

14. The computerized system of claim 12, wherein
the visualization presented to the user comprises a virtual representation or an augmented representation of a body part of the user, and
the virtual representation or the augmented representation depicts the body part of the user acting with a greater activation force or moving with a larger degree of rotation than a reality-based activation force or a reality-based degree of rotation of the body party of the user.

15. The computerized system of claim 13, wherein
the at least one computer processor is further programmed to determine, based on one or both of: the plurality of neuromuscular signals and information derived from the plurality of neuromuscular signals, deviation information from the at least one target neuromuscular activity state, and
the feedback provided to the user comprises a visualization based on the deviation information.

16. The computerized system of claim 15, wherein the deviation information is derived from a second plurality of neuromuscular signals processed by the at least one computer processor.

17. The computerized system of claim 15, wherein
the at least one computer processor is further programmed to predict an outcome of the athletic movement or the therapeutic movement performed by the user based, at least in part, on the deviation information, and
the feedback provided to the user comprises an indication of the predicted outcome.

18. A method performed by a computerized system for providing feedback to a user based on neuromuscular signals sensed from the user, the method comprising:
receiving a plurality of neuromuscular signals sensed from the user using a plurality of neuromuscular sensors arranged on one or more wearable devices worn by the user;
processing the plurality of neuromuscular signals using one or more inference or statistical models; and
providing feedback to the user based on one or both of: the processed neuromuscular signals and information derived from the recorded neuromuscular signals,
wherein the feedback provided to the user comprises visual feedback that includes information relating to one or both of:
a timing of an activation of at least one motor unit of the user, and
an intensity of the activation of the at least one motor unit of the user.

19. The method of claim 18, wherein the visual feedback provided to the user comprises is provided within an augmented reality (AR) environment generated by an AR system or a virtual reality (VR) environment generated by a VR system.

20. The method of claim 18, wherein the feedback provided to the user comprises auditory feedback or haptic feedback or auditory feedback and haptic feedback that relates to one or both of:
the timing of the activation of the at least one motor unit of the user, and
the intensity of the activation of the at least one motor unit of the user.
